(19) United States
(12) Patent Application Publication
(10) Pub. No.: US 2023/0210506 A1
(43) Pub. Date: Jul. 6, 2023

(54) NEEDLE-ASSISTED AUTOMATED INSERTION AND EXTRACTION OF IMPLANTS

(71) Applicant: Biorasis Inc., Storrs, CT (US)

(72) Inventors: Fotios Papadimitrakopoulos, West Hartford, CT (US); Allen Legassey, Mansfield, CT (US); Joon-Sung Kim, Vernon, CT (US); Jun Kondo, Hartford, CT (US); Faquir Jain, Storrs, CT (US)

(21) Appl. No.: 17/928,834

(22) PCT Filed: Apr. 1, 2021
(86) PCT No.: PCT/US2021/025450

§ 371 Date: Nov. 30, 2022

Related U.S. Application Data
(60) Provisional application No. 63/032,924, filed on Jun. 1, 2020.

(51) Int. Cl. A61B 10/06; A61B 17/34

(52) U.S. Cl. CPC A61B 10/06; A61B 17/3468; A61B 2017/00349

(57) ABSTRACT

Disclosed herein is a percutaneous catheter apparatus, comprising two nested needles; and an inner plunger; which is guided as a catheter to the tissue surrounding a hard implant to actuate and deploy a pair of sharp-tip needle-forceps that perform two concentric cuts, circularly spaced 90-degree apart from each other, to complete a 360 degree bore around the implant before squeezing to arrest and extract the implant, together with its surrounding tissue.

### CROSS REFERENCE TO RELATED APPLICATIONS

This application claims the benefit of U.S. Application No. 63/032,924 filed on Jun. 01, 2020 which is incorporated herein by reference in its entirety.

### STATEMENT OF FEDERAL SUPPORT

This invention was made with government support under CDMRP W81XWH-16-C-0069 awarded by the Congressional Directed Medical Research Program. The government has certain rights in the invention.

### BACKGROUND

This disclosure relates to a needle-assisted automated device for insertion and extraction and insertion of implants. More particularly, this disclosure relates to a needle-assisted automated device for the efficient retrieval of hard miniaturized implants. Recently, there is great interest for highly miniaturized implants composed of small CMOS (complementary metal oxide semiconductor) integrated circuits. Such CMOS circuits are typically made of silicon wafers and are cut in small, elongated chips so that they can go through small hypodermic needles. These CMOS-based implants can be configured as sensors (i.e. for continuous glucose monitoring and metabolic functions), smart ratio-frequency tags to enhance care, safety and efficiency through increased RFID visibility and automation, and remote point of care diagnostic tools and other usages.

Small CMOS integrated circuits, aside from their extreme hardness in comparison to biological tissues, can be particularly brittle in an elongated form. CMOS-based implants may also be passivated with brittle coatings (i.e., glass, SiN, etc.) and can also be outfitted with powering coils, sensing electrodes, and other miniaturized components attached to them. Such miniaturized implants may be encased within soft polymeric or hydrogel coatings to enhance biocompatibility and also afford localized delivery of small amounts of drugs (typically called tissue response modifiers) to suppress foreign body response and fibrosis.

The hardness, brittleness and structure complexity of such miniaturized implants render their removal particularly challenging at the end of their useful lifetime. This is further exacerbated by their small size and their possible adhesion to their surrounding tissue. Localized surgery is a safe option but it is generally expensive and may create extensive trauma to the host. Based on this, there is a significant interest in the community for enabling stereotactically-guided biopsy retrieval of these miniaturized implants with minimal trauma to the host and significant cost savings.

Stereotactically-guided biopsy-based retrieval has been developed as a great asset in obtaining biopsy specimens for further analysis. This is attributed to the enhanced accuracy of modern imaging techniques that allow physicians to locate lesions with ever-increasing precision. There is a plethora of modern biopsy retrieval tools, which are typically developed for soft-tissue excision. When however, a hard and/or brittle object is involved (i.e. implant) together with soft-tissue excision around this object, things get significantly more challenging.

Boring around the implant must be very carefully performed to avoid breaking complex implants or damaging the boring tool. Moreover, boring around the implant is challenged by strong implant-tissue adherence. This leads to pushing and misaligning the implant with respect to the boring tool, which further complicates extraction. Accordingly, larger bore core catheters might be needed to safely bore around the implant and dislodge it from its surrounding tissue, using core needle biopsy. Aspiration- or vacuum-assisted biopsy has the additional feature that can gently arrest the implant and safely guide it out. Because of tissue-implant interactions, both radius for core needle and vacuum assisted biopsy must be large in order to avoid damaging the tool. Consequently, because of the large radius both of these techniques could lead to excessive subcutaneous tissue uptake and unnecessary skin cavitation.

In order to overcome these disadvantages it is desirable to develop a needle-assisted device for the efficient retrieval of hard miniaturized implants from the body of a living being.

### SUMMARY

Disclosed herein is a percutaneous catheter apparatus, comprising two nested needles; and an inner plunger; which is guided as a catheter to the tissue surrounding a hard implant to actuate and deploy a pair of sharp-tip needle-forceps that perform two concentric cuts, circularly spaced 90-degree apart from each other, to complete a 360 degree bore around the implant before squeezing to arrest and extract the implant, together with its surrounding tissue.

Disclosed herein too is a methodology to aid the operator of a percutaneous catheter tool to superimpose the real time shape and position of its sharp-tip needle-forceps to tomographic obtained images, in order to facilitate the safe boring of the tissue surrounding a hard implant prior of extract it together with the arrested implant.

Disclosed herein too is a device that is directed to a percutaneous insertion and extraction catheter tool that places an implant at a predetermined location and extracts it out after some period, comprising two nested catheters and an inner plunger; a handheld tool that holds the two nested catheters and the inner plunger; a battery powered controller unit with a flat panel display; and an ultrasound imaging system that guides the catheter tool to the desired location. The two nested catheters comprise an outer catheter and inner catheter; wherein the tip of the outer catheter comprises of a sharp-point needle with an elongated step at half height; and wherein the tip of the inner catheter comprises of a sharp-point needle with a triangular serrated cut below, followed by a tubular segment and then a thin bottom segment that is bent upwards to define a flex-operated hinge.

The plunger resides within the inner catheter. The flex-operated hinge of the inner catheter is operated by the sliding in and out of the plunger. The outer and inner catheter create a sharp-tip needle-forceps. The sharp-tip needle-forceps are open when the inner plunger is withdrawn past the flex hinge. The sharp-tip needle-forceps are partially closed when the inner plunger is halfway withdrawn over the flex hinge. The sharp-tip needle-forceps are fully closed and nested within the outer catheter when the inner plunger is directly over the flex hinge.

The outer catheter, inner catheter, and inner plunger are independently actuated with three stepping motors housed within the handheld tool, said inner plunger is attached first to a force gauge and then connected with its stepping motor to obtain force measurement. The outer and inner catheter can rotate 90 degrees with sliding a bar attached to the handheld tool, and the handheld tool has linear bar markers that indicate the relative travel of all said outer catheter, inner catheter, and inner plunger.

The controller unit displays on its screen the relative travel of all said outer catheter, inner catheter, and inner plunger in absolute travel, percentage and linear bar format, and the controller unit has predetermined translation steps for the outer catheter, inner catheter, and inner plunger for functions including, deploy the sharp-tip needle-forceps, perform a concentric double-90-degree cut to bore the tissue around the implant, perform a controlled squeeze around the bored tissue to arrest the implant, deploy a nested implant at the desired place with the desired orientation, operate a foot control unit that manually controls and alters all said predetermined translation steps, where the controller unit displays in real-time the shape and configuration of the sharp-tip needle-forceps.

The real-time shape and configuration of the sharp-tip needle-forceps are juxtaposed with the ultrasound images to facilitate the operator. These ultrasound images display multiple resonances underneath both said implant and catheter indicate alignment of these objects in the ultrasound imaging-plane. The ultrasound multiple resonances underneath the implant aid the operator to accurately determine the site of percutaneous catheter insertion, and the controller unit permits operation selected from manual, semi-automatic, and automatic mode chosen by the operator, and the operation is controlled by two forward and backward buttons located at the sides of the handheld unit that actuate the various steps of the manual, semi-automatic, and automatic mode chosen by the operator.

### BRIEF DESCRIPTION OF THE FIGURES

FIG. 1(A) is an exemplary depiction of the device that comprises a retrieval unit, a battery powered controller unit with a flat panel display and an ultrasound imaging system that guides the catheter tool to the desired location;

FIG. 1(B) is an exemplary depiction that illustrates the nested catheter probe configuration of the retrieval unit together with its needle-based “micro-forceps” action in its fully open state;

FIG. 1(C) is an exemplary depiction that illustrates the nested catheter probe configuration of the present invention together with its needle-based micro-forceps action in its half open (squeeze) state;

FIG. 1(D) is an exemplary depiction that illustrates the nested catheter probe configuration of the present invention together with its needle-based micro-forceps action in its fully-retracted state, fitting withing the outer catheter;

FIG. 2(A) is an exemplary depiction of a first pair of cuts made in tissue by the retrieval tool,

FIG. 2(B) is another exemplary depiction of a second pair of cuts made in tissue by the retrieval tool; 90-degree rotated from the cut in FIG. 2(A) to complete 360-degree boring around the implant;

FIG. 3(A) depicts the securing of the implant in the retrieval unit;

FIG. 3(B) depicts the withdraw of the catheter, exposing the implant beyond the catheter towards and into the tissue of a living being;

FIG. 3(C) depicts retraction of the inner catheter to prepare for release of the implant into the tissue;

FIG. 3(D) depicts the release of the implant into the tissue from the retrieval unit;

FIG. 4 shows in a step-by-step manner, using a transparent gelatin matrix, how boring and extraction is performed for a hard implant (dark) and its surrounding matrix (gray), respectively;

FIG. 5 shows close up images of the extracted implant (dark) within its surrounding matrix (gray) in and out of the needle-based micro-forceps;

FIG. 6 shows a soft gelatin plug bored and extracted from a transparent gelatin matrix using the needle-based micro-forceps;

FIG. 7(A) is a photomicrograph that shows 12 MHz ultrasound images of an implant and a nearby extraction catheter situated between the ulna and radius bones of the chicken wing;

FIG. 7(B) is another photomicrograph that shows 12 MHz ultrasound images of an implant and a nearby extraction catheter situated between the ulna and radius bones of the chicken wing;

FIG. 7(C) is yet another photomicrograph that shows 12 MHz ultrasound images of an implant and a nearby extraction catheter situated between the ulna and radius bones of the chicken wing;

FIG. 7(D) is yet another photomicrograph that shows 12 MHz ultrasound images of an implant and a nearby extraction catheter situated between the ulna and radius bones of the chicken wing;

FIG. 8(A) is a schematic depiction of the multiple resonances underneath both implant and catheter that may be used to indicate alignment of these objects in the ultrasound imaging-plane;

FIG. 8(B) is a photomicrograph that corresponds to the schematic depiction of FIG. 8(A) of the location of both implant and catheter that may be used to indicate alignment of these objects with the ultrasound imaging-plane. In FIGS. 8(A) and 8(B), the implant may be seen to be close to the needle(s);

FIG. 8(C) is a schematic depiction of the multiple resonances underneath only implant and not the catheter that may be used to indicate lack of alignment of these objects in the ultrasound imaging-plane;

FIG. 8(D) is a photomicrograph that corresponds to the schematic depiction of FIG. 8(C) of the location of both implant and catheter that the lack of multiple resonances underneath the catheter may be used to indicate mis-alignment of these objects with the ultrasound imaging-plane;

FIG. 9(A) shows 12 MHz ultrasound images for boring and extraction of an implant within the subcutaneous tissue of a chicken wing in a step-by-step manner;

FIG. 9(B) identifies all objects observed in the ultrasound images of FIG. 9(A);

FIG. 10(A) shows an exemplary method of how the multiple resonances underneath a hard implant can be used to identify and mark the percutaneous injection site and the orientation of the needle-based micro-forceps extraction catheter;

FIG. 10(B) shows an exemplary method of how the multiple resonances underneath the hard implant of FIG. 10(A) can be used to identify and mark the percutaneous injection site and the orientation of the needle-based micro-forceps extraction catheter;

FIG. 10(C) shows an exemplary method of how the multiple resonances underneath the hard implant of FIG. 10(A) can be used to identify and mark the percutaneous injection site and the orientation of the needle-based micro-forceps extraction catheter;

FIG. 10(D) shows an exemplary method of how the multiple resonances underneath the hard implant of FIG. 10(A) can be used to identify and mark the percutaneous injection site and the orientation of the needle-based micro-forceps extraction catheter;

FIG. 11(A) illustrates an exemplary embodiment of the nested catheter set (needles and plunger from the FIGS. 1(A) - 1(D)) for insertion or extraction;

FIG. 11(B) illustrates an exemplary embodiment, of the insertion/extraction tool;

FIG. 11(C) illustrates an exemplary embodiment of the assembled extraction tool with open lid;

FIG. 11(D) illustrates an exemplary embodiment of the assembled extraction tool with open lid;

FIG. 12(A) illustrates an alignment plane in an exemplary embodiment of the rotary mechanism for the needle-based micro-forceps to afford 360° boring around the tissue surrounding the implant;

FIG. 12(B) illustrates indicates one edge of the implant in another exemplary embodiment of the rotary mechanism for the needle-based micro-forceps to afford 360° boring around the tissue surrounding the implant;

FIG. 13(A) illustrates an exemplary embodiment of the visual gauge mechanism to assess the relative translation of three components (both the needles and the plunger) of FIGS. 1(A) - 1(D), which define the shape of the needle-based micro-forceps;

FIG. 13(B) depicts an isometric view of the insertion extraction tool with color coded indicator bars marked C (catheter), T (tool) and P (plunger) for the positions of both the needles and the plunger;

FIG. 13(C) depicts an exemplary embodiment of the color-codded indicator bars that are confined between the motor frame and outer case of the insertion/extraction tool;

FIG. 13(D) depicts and exemplary embodiment of the insertion/extraction tool where the positions of the needles and the plunger can be gauged by looking through the three openings on the top of the case;

FIG. 14(A) illustrates an exemplary embodiment (as seen from one side) of the hand-tool operating buttons equipped with a hand rest and haptic recognition for forwards and backwards motion;

FIG. 14(B) illustrates another exemplary embodiment (as seen from the opposite side of the FIG. 14(A)) of the hand-tool operating buttons equipped with a hand rest and haptic recognition for forwards and backwards motion;

FIG. 15(A) illustrates the battery powered control unit for motorized insertion and extraction hand-tool;

FIG. 15(B) illustrates representative captured images of the cover flat panel display describing calibration;

FIG. 15(C) illustrates representative captured images of the cover flat panel display detailing implant insertion;

FIG. 15(D) illustrates representative captured images of the cover flat panel display detailing boring/extraction;

FIG. 16 illustrates the foot-control-unit to enable manual adjustments of the catheter (needle 14), tool (needle 12) and plunger 16 of the nested catheter micro-forceps.

FIG. 17 illustrates the distinct steps together with the relative plunger, tool and catheter displacement (shown in the Line Bar Target) needed to impart the desired shape changes to the overall needle-based micro-forceps (Tool) for the controlled implant insertion according to FIG. 3.

FIG. 18 illustrates the distinct steps together with the relative plunger, tool and catheter displacement (shown in the Line Bar Target) needed to impart the desired shape changes to the overall needle-based micro-forceps (Tool) for the controlled implant extraction according to FIG. 1 and FIG. 2.

FIG. 19 illustrates the three-day visual verification to the operator that the insertion/extractor tool functions properly;

FIG. 20 illustrates how the plunger is equipped with a force sensing gauge to measure the needle-based micro-forceps grabbing force;

FIG. 21(A) illustrates a highly-integrated tool, where ultrasound images, relative translation bars, and expected shape of the needle-based micro-forceps are real-time displayed on a high resolution flat-panel display right on top of the percutaneous catheter; and

FIG. 21(B) show how the tool is operated from an ergonomic rotary dial equipped with haptic interface for translation speed and force feedback.

### DETAILED DESCRIPTION

Disclosed herein is a device and a methodology for percutaneous insertion and extraction of hard and brittle miniaturized implants in the bodies of living beings. The device may also be used for removal of tissue for biopsies and autopsies in living beings. This device uses a retrieval tool that is appropriately shaped at its distal end to be able to bore around an implant in order to dislodge it from its surrounding tissue. The device comprises a specialized, nested percutaneous biopsy tool that can deploy a needle-shaped micro-forceps to be used as a means of boring into the tissue (of a living being) and arresting the implant. In order to retrieve or to insert an implant, imaging may be used to assess the depth and orientation of both the implant together with the retrieval tool (and the spatial configuration of the retrieval tool) inside the body of a living being. The imaging is used to manipulate the tool relative to the implant and to retrieve the implant.

Both the retrieval tool along with the imaging screen are contained in a single hand held device. This is advantageous because the imaging screen provides unparalleled situational awareness to the medical professional that operates the tool in either a fully-automated, semi-automated or manual mode during the insertion and extraction of tissue or an implant. The ability to display all such information at the site of percutaneous injection is geared to utilize the exceptional hand-skills of medical professionals, who might not be trained in the field of radiology (i.e., skilled to operate a tool while watching at a screen far away from the tool). This enables easy training of health practitioners, which facilitates broadening the pool of suitable candidates that can be trained on how to use the device.

The device is particularly suited for stereotactically-guided biopsy retrieval of brittle and complex miniaturized implants together with its surrounding tissue that can be used for further biopsy studies. While the tool described herein can be employed with majority of stereotactic guiding and imaging methods, particular interest has been directed to ultrasound guidance. This is because high frequency ultrasound (typically in the range of 7-to-50 MHz) is ideally suited for skin imaging with a penetration depth of 3 to 25 millimeters (mm), preferably 5 to 15 mm. High frequency ultrasound can easily assess the exact thickness of epidermis and dermis, as well as the size and depth of the subcutaneous tissue, where miniaturized implants reside and should be implanted.

This invention relies strongly to real-time data fusion, where ultrasound images need to be juxtaposed alongside an image of the mechanical motion of the retrieval tool (that continuously changes due to the three-dimensional (3D) configuration of the retrieval tool while in motion). The success of this device relies on the ability of providing accurate situational awareness to the medical professional that operates the retrieval tool and the ultrasound probe. In typical ultrasound imaging, the medical professional spends a considerable amount of his/her time looking at the screen where the ultra-sound image is projected. In doing so, the medical professional is trying to decipher based on the limited resolution and image obstructions/irregularities what he/she is actively seeing. By providing the three-dimensional (3D) local configurations of the tool, the operator can be readily guided in deciphering the ultrasound image and actively coached on how to prevent accidental cracking of implant during its arrest and removal.

Based on this, it becomes apparent that data fusion becomes important in the proper operation of the tool described. Typically, all data are projected at one or two monitor(s) that lay 90 degrees away from the percutaneous site. This requires trained radiologists that have developed special skills to watch one or more monitors, while moving the biopsy tool without visual contact. Combining the obtained ultrasound images together with the displacement-predicted 3D configuration and haptic-force feedback on the biopsy retrieval tool at the percutaneous site is currently deemed paramount. This is because all medical professionals possess great eye-to-hand coordination. Combining ultrasound imaging, tool shape, and haptic force feedback at the site of implantation can enable greater adoption to health practitioners and broaden the pool of suitable candidates to be trained on how to use the tool described in this invention.

Disclosed herein is a percutaneous insertion and extraction catheter tool (hereinafter a retrieval tool) that places an implant at a predetermined location and extracts it out after some period. The retrieval tool comprises two nested catheters (which end in razor sharp needles, also known as jaws) and an inner plunger, a handheld tool that holds the two nested catheters and the inner plunger, a battery powered controller unit with a flat panel display, and an ultrasound imaging system that guides the catheter tool to the desired location.

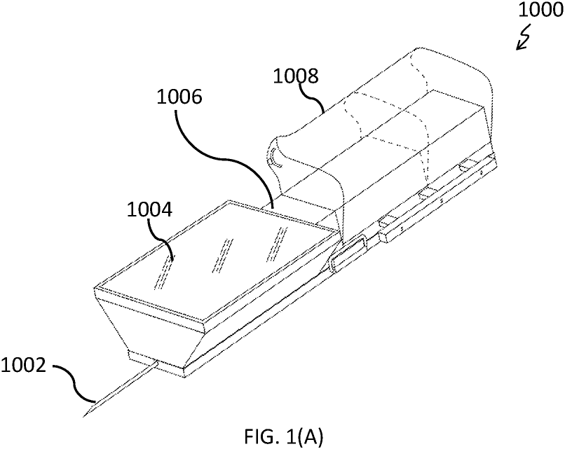

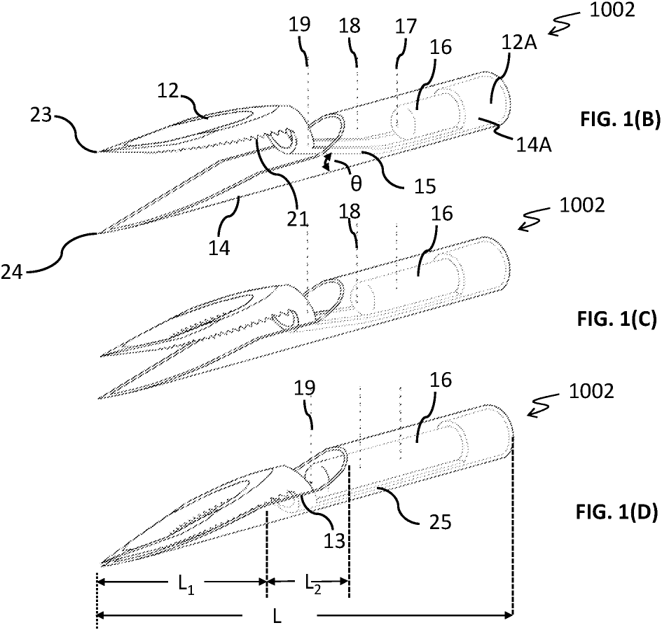

With reference now to FIG. 1(A), the device 1000 comprises a handheld tool 1008 with retriever unit 1002. a battery powered controller unit 1006 with a flat panel display 1004. From the FIG. 1(A) it may be seen that the handheld tool 1008 (which can be held by hand) lies on the opposite side of the flat panel display 1004 from the retriever unit 1002. The display lies atop the battery powered controller unit 1006.

The retriever unit 1002 will now be detailed with reference to the FIGS. 1(B), 1(C) and 1(D). FIG. 1(B) is an exemplary depiction that illustrates the of the retrieval unit together with its needle-based “micro-forceps” action in its fully open state. FIG. 1(C) is an exemplary depiction that illustrates the retrieval unit with its needle-based micro-forceps action in its half open (squeeze) state. FIG. 1(D) is an exemplary depiction that illustrates the retrieval unit together with its needle-based micro-forceps action in its fully-retracted state, fitting within the outer catheter. With reference now to the FIG. 1(B), the retrieval unit 1002 that is affixed to the device and that may be manipulated by the battery powered controller unit 1006.

FIG. 1(B) depicts the configuration of the retrieval unit 1002 along with its needle-based “micro- forceps” action. This retriever unit comprises two opposed needles 12 and 14 that are in operative communication with a plunger 16. Operative communication includes mechanical communication, pneumatic communication, electrical communication, optical communication, or a combination thereof. The plunger 16 is a solid plunger. The needles 12 and 14 can function as a cutting tool as well as a pair of jaws to cut out a section of tissue (that may or may not contain an implant) (not shown). In an embodiment, the needles 12 and 14 are coaxially mounted about the plunger 16. As shown in FIGS. 1(B), (C) and (D), the two co-axial needles 12 and 14 are appropriately shaped to create the two opposing jaws of a “micro-forceps”. The term “micro-forceps” therefore refers to the opposing jaws of the two coaxial needles 12 and 14 which move towards one another to capture a sample or an implant and move away from one another to release it. The tips 23 and 24 of the needles 12 and 14 respectively represent the distal end of the retrieval unit 1002. while the opposite end of the needles (represented by numerals 12A for the needle 12 and 14A for the needle 14) represent the proximal end of the retrieval unit 1002. The proximal end of the retrieval unit 1002 contacts the battery powered controller unit 1006. which will be detailed later.

A portion of the coaxial needle 14 also functions as a catheter 14A that contacts the battery powered controller unit 1006. This will be discussed in detail later. The catheter portion 14A of the coaxial needle may be a 10 gauge to 34 gauge needle, a 14 gauge to 28 gauge needle, preferably a 15 gauge to 22 gauge needle. The catheter portion 14A forms the outer shell of the retrieval unit in which the catheter portion 12A of the needle 12 can slide back and forth. The plunger 16 also slides back and forth in the catheter portion 14A. In an embodiment, the plunger 16 slides inside the catheter portion 12A of the needle 12 (which is coaxially located inside the catheter portion 14A).

The catheter portion 14A of the needle 14 is tubular with the outermost distal portion having a tip 24 that is shaped in such manner to possess a sharp shallow cut that ends to a very acute point. The tip 24 of the needle 14 is shaped in such manner to possess a sharp shallow cut that ends to a very acute point. At the half diameter height of the needle 14, a straight cut 13 is situated to allow the inner needle 12 to flex upwards with no hinderance.

With reference to the FIG. 1(D), the needle has a total length L of which a first portion L₁ may be sectioned off (have one portion of the tubular section removed) so that it can be used to penetrate tissue more easily and cut out a section of the tissue for examination. As may be seen in the FIG. 1(D), the section of the needle 14 of length L₁ has a smaller circumference than the section of length L₂. The section of length L₂ has the straight cut 13. The circumference of the needle reduces to a sharp point from the straight cut 13 to the tip 24.

The ratio of L₁ to L₂ can vary from 10:1 to 1:10, preferably 5:1 to 1:5. The ratio of the sum of L₁ and L₂ to L (the entire length of the needle) can vary from 1:1.5 to 1:5, preferably 1:2 to 1:4.

The needle 12 (which opposes needle 14) contacts catheter 12A. Catheter 12A is coaxial about catheter 14A and can slide into catheter 14A during assembly. It stays in a fixed position with respect to the catheter 14A inside the catheter 14A when installed. The needle 12 represents the distal end of the catheter 12A and ends in a very acute point 23 (also called the tip 23). The portion of the needle 12 away from the tip 23 towards the proximal end of the catheter 12A is serrated with teeth 21 disposed on the jaw to increase friction with the implant and its surrounding soft tissue during capture and extraction. As may be seen in the FIGS. 1(B) and 1(C), the opposing jaw of the needle 12 (from the needle 14) has the serrated edge. The catheter 12A is machined to have a thin flat portion 25 at the bottom towards the proximal end of the retriever tool.

Between the serrated portion with teeth 21 and the thin flat portion 25 at the bottom of the needle 12 lies a grabber 15, which comprises a section of material that is bent away from the thin flat portion 25. The plane of the grabber 15 is at an angle θ with respect to the plane of the thin flat portion 25. The angle θ may vary from 5 degrees to 70 degrees, preferably 10 to 50 degrees. The material is bent away in such a manner that causes the needle 12 to move away from the needle 14 when the plunger 16 is moved towards the proximal end of the retrieval unit 1002 and to move towards the needle 14 when the plunger 16 is moved towards the distal end of the retrieval unit 1002. This arrangement of the grabber 15 with respect to the thin flat portion 25 and the activation of the jaws (needle 12 and needle 14) by the plunger 16 may be referred to as a flex operated hinge. The grabber 15 may optionally have a grooved surface to permit motion for the plunger 16 that activates the needle 12 to move towards or away from the needle 14.

By applying a controlled upward bend to the thinner portion of the grabber 15 the “open micro-plier configuration” is mechanically “stored” in the inner jaw. Micro-plier activation takes place with the help of an inner rod 16, herein referred as to as the plunger 16. As shown in FIGS. 1(A), 1(B) and 1(C), advancing the plunger 16 forwards (from position 17 to 18 and 19) causes the jaws of the micro-forceps (the needles 12 and 14) to gradually close as the grabber 15 flattens to be in the same plane as the thin flat portion 25. When the plunger 16 is extended all the way forwards to the position 19, the needles 12 and 14 contact each other and are fully closed (See FIG. 1(C)). On the other hand, when the plunger 16 is extended all the way back to the position 17, the needles 12 and 14 open and are as far apart from each other as permitted by the grabber 15 (See FIG. 1(A)). In this fashion, both the grabber 15 and the plunger 16 nest nicely inside the catheter 14 to ease percutaneous insertion.

It is to be noted that the needle 12 and the catheter 12A are interchangeably referred to as the catheter, while the needle 14 and the catheter 14A are interchangeably referred to as the tool. The catheter 12A, the catheter 14A and the plunger 16 are referred to as the nested catheter(s). The nested catheters would therefore also refer to the needle 12, needle 14 (both of which form the jaws for insertion and extraction) along with the plunger 16.

In summary, the two nested catheters (catheter 12A and 14A) that form the retrieval tool comprise an outer catheter (14A) and inner catheter (12A), wherein the tip of the outer catheter comprises of a sharp-point needle 14 with an elongated step at half height, and the tip of the inner catheter 12 comprises of a sharp-point needle with a serrated edge 21. The inner catheter 12A narrows to a needle 12 with tip 23 via a thin flat portion 25 and a grabber 15. The catheter 14A narrows to a needle 14 with tip 25. The outer and inner catheter 14A and 12A respectively create a sharp-tip needle-forceps at the distal end of the retrieval unit 1002. The plane of the grabber 15 is inclined at an angle θ to the plane of the thin flat portion 25 thus creating a flex operated hinge that is activated by the motion of the plunger 16. The plunger 16 is a solid that resides within the inner catheter 12A and the flex-operated hinge of the inner catheter is operated by sliding the plunger 16 in and out from the positions 17 to 19. In an embodiment, the said sharp-tip needle-forceps (12 and 14) are open when the inner plunger is withdrawn past the flex hinge; are partially closed when the inner plunger is halfway withdrawn over the flex hinge; and are fully closed and nested within the outer catheter, when the inner plunger is directly over the flex hinge.

As previously detailed, performing biopsies on the tissue surrounding the implant is highly desirable. Typical biopsy methods (i.e. vacuum biopsy, percutaneous biopsy, punch biopsy, etc.) are conducted in the absence of a hard implant, which can either damage the tool or break the implant. Also, in the case that the hard implant is first removed, the site-of-interest might be substantially altered for subsequent biopsy excisions. The ideal scenario is to remove the implant together with its surrounding tissue.

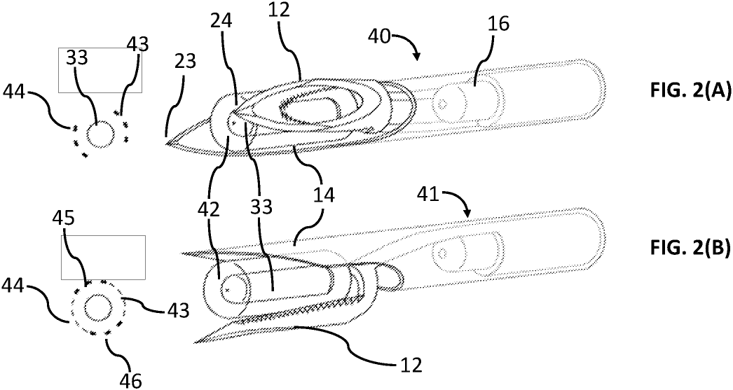

The needle-based “micro-forceps” tool described in this invention can perform careful boring around a variety of soft (normal tissue), medium and relatively hard matter (i.e. lesions, scar tissue, calcified tissue, etc.). The example described in the FIGS. 2(A) and 2(B) uses the retrieval tool 1002 to perform two coaxial cuts (43 and 44) around a hard and brittle implant 33. These cuts are offset 90° with respect to each other. FIG. 2(A) is an exemplary depiction of a first pair of cuts made in tissue by the retrieval tool, while the FIG. 2(B) is another exemplary depiction of a second pair of cuts made in tissue by the retrieval tool. These figures illustrate how the needle-based micro-forceps enable 360° boring around the tissue (e.g., such as that surrounding an implant).

As shown in FIG. 2(A), by extending the needles 12 and 14 slightly wider than the diameter of the implant 33 the bore 42 diameter is set. Subsequently, in the first motorized travel along the length of the implant, two 90 degree cuts 43 and 44 are performed on opposing sides by the razor-sharp edges situated left and right of the “micro-forceps” tips 23 and 24 of the needles 12 and 14, respectively. Each cut covers an arc of 90 degrees. Since there are two needles 12 and 14 each making a cut of 90 degrees, the total sum of the arcs made curing the first cut is 180 degrees. The second cut (detailed below) is another arc of 180 degrees for a total circumferential arc (first cut + second cut) of 360 degrees.

The next step is to withdraw back the jaws (needles 12 and 14) to the starting position and perform a co-axial 90° rotation for both the needles 12 and 14. On the next motorized travel along the length of the implant, two additional 90 degree cuts 45 and 46 are performed, to complete the cylindrical boring (FIG. 2(B)). Since the surrounding tissue around the implant is now completely bored, a mild squeeze of the needles 12 and 14 permits both the implant 33 and its surrounding tissue 42 to be retrieved in their original configuration.

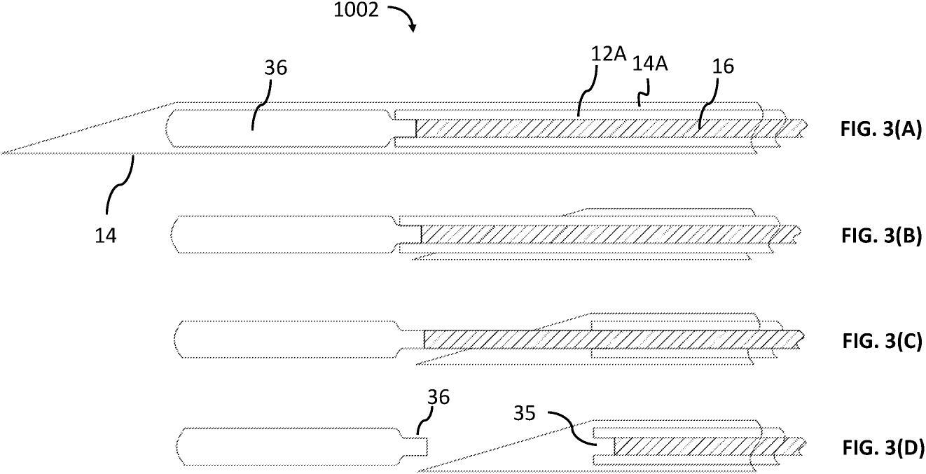

FIGS. 3(A), 3(B), 3(C) and 3(D) illustrates another embodiment of this invention, which includes the insertion of tissue or an implant into the body of a living being. FIG. 3(A) depicts the securing of the implant 36 in the retrieval unit 1002. The retrieval unit is modified for this embodiment as detailed below. FIG. 3(B) depicts the exposing of the implant into the tissue of a living being by withdrawing the catheter 14A backwards. FIG. 3(C) depicts retraction of the catheter 12A to prepare for release of the implant 36 into the tissue. FIG. 3(D) depicts the release of the implant 36 into the tissue from the retrieval unit. These figures illustrate the injection sequence of a miniaturized implant when the implant is originally nested within the outer catheter. This sequence ensures that implant rotating does not occur during device implantation.

This embodiment pertains to reshaping the nested catheter configuration with two co-axial needles 12 and 14 and the inner plunger 16 as a highly accurate implanter for the controlled insertion of miniaturized implants 36. Some of these implants are sensitive in terms of their placement depth and relative orientation with respect to the skin. For miniaturized sensors, placement depth and orientation greatly can strongly affect the coupling power (e.g., light, radiofrequency (RF), ultrasound, and the like) from external devices that power and communicate with the implant.

For this, the tip of the tool (the inner needle 12) or the plunger 16 can be equipped with a notch 35 (or other restriction or arrest mechanism) that prevents implant rotation during implantation. FIG. 3(D) depicts this notch 35 at the tip of the inner needle 12. This configuration allows sensitive implants 36 to be safely packaged, sterilized, transported and stored within the insertion needle 14 (slightly behind its tip). By cutting the tips of the notch 35 slightly lower than 90°, adequate gripping power can be exerted to the implant end 36, to prevent unwanted release from the tip of the retrieval tool 1002.

The implant deployment sequence is shown in FIGS. 3(A) to 3(D). When the inserter tip reaches the desired implantation site, the catheter 14A retracts as seen in the FIGS. 3(A) and 3(B), while the catheter 12A and the plunger 36 remain stationary. Next, in the FIG. 3(C), the catheter 12A retracts while the plunger 16 remains stationary. This action controllably releases the implant 36 at the desired location. Retraction of the plunger 16 in the FIG. 3(D), nests back both catheter 12A and the plunger 16 within the outer catheter 14A, and the tool is safely pulled away having released the implant 36 into the desired region of the tissue.

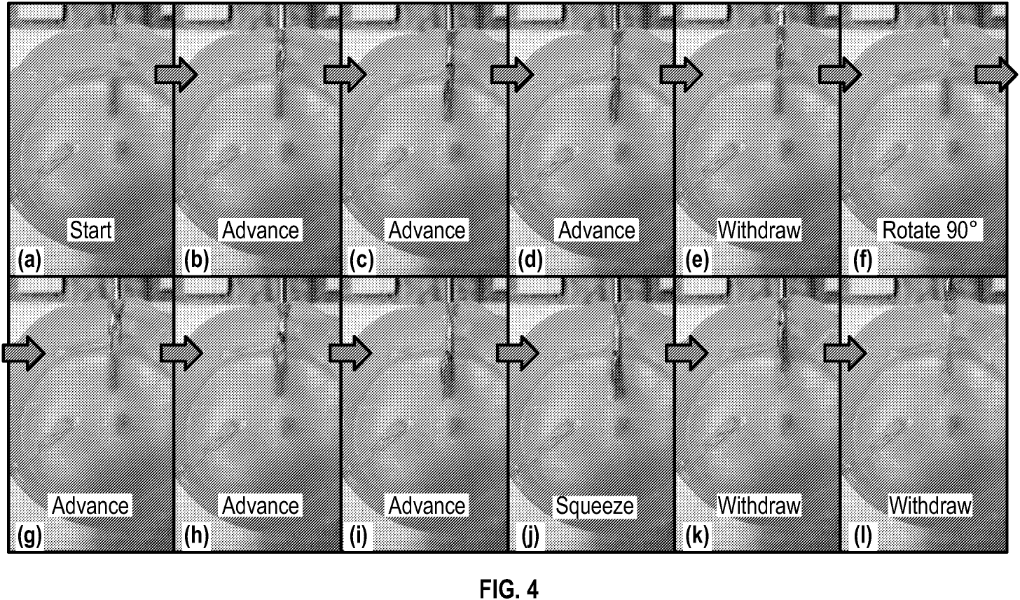

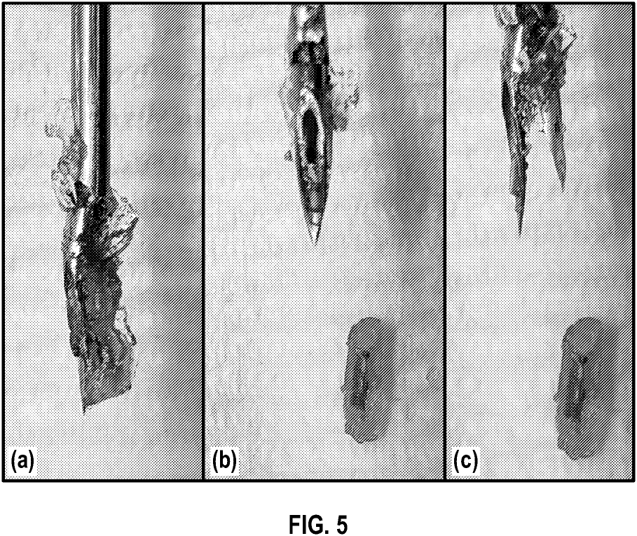

FIGS. 4 and 5 illustrate a sequence of captured images from a boring and extraction video using the device 1000 detailed herein. The sharp needle tips 23 and 24, when are mechanically translated around the implant, they first perform an upper and lower cut (FIGS. 4b-d). Subsequently, the needle tips retreat (FIG. 4e), are then rotated 90° (FIG. 4f), and a second cut is performed on the left and right side (FIGS. 4g-i) to complete the boring process. Finally, the implant and its surrounding red gelatin matrix is easily extracted by a gentle squeeze of the needles (FIG. 4k) and withdrawn (FIG. 4l). Close up images of the bored and extracted implant together with its surrounding red-colored gel are shown in FIG. 5 within the “micro-forceps” (FIG. 5a) and released (FIGS. 5b, c).

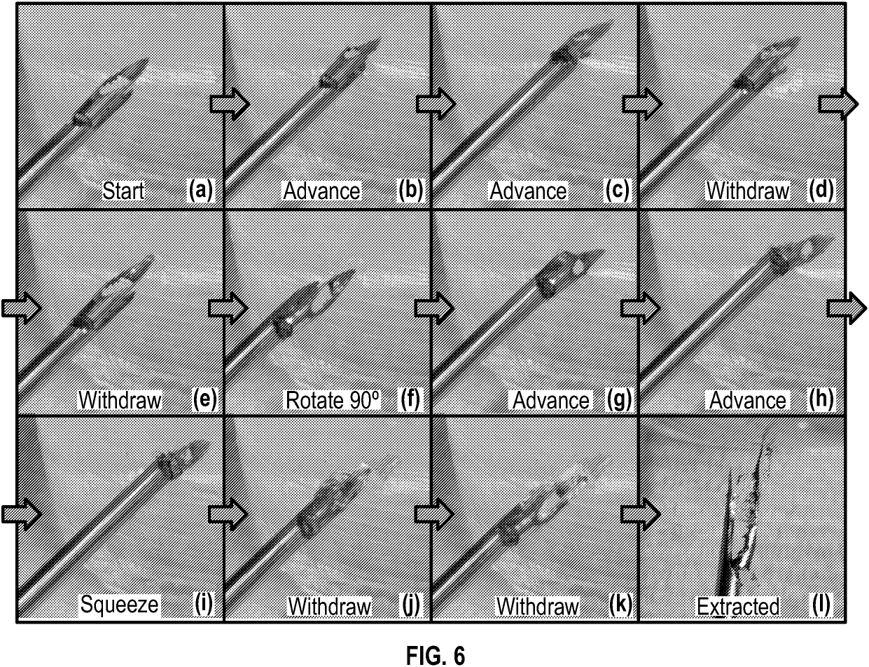

The sequential captured images in FIG. 6 also illustrates that the boring concept can be also performed in the absence of a hard implant. Here the sharp catheter and grabber needle tips (see needle tips 23 and 24 from FIGS. 1(A) - 1(D)), when mechanically advanced into the gelatin they cut an upper and lower cut (FIGS. 6b, c). After withdrawal (FIGS. 6d, e) and 90° rotation (FIG. 6f), a second cut is performed on the left and right side (FIGS. 6g, h), which completes the boring process. Finally, the bored gelatin is easily extracted (FIG. 6l) by a gentle squeeze of the needle(s) 12 and 14 (FIG. 6i) and withdraw (FIGS. 6j, k). This is particularly useful for extracting subcutaneous tissue from a nearby location that can serve as control for various histopathology studies.

It is desirable to note another salient feature of the “micro-forceps” induced boring. This has to do with boring mechanically tough biological structures such as scar- and calcified-tissues. The cutting force applied by the opposing “micro-forceps” sharp tips is highly balanced as opposed to other core biopsy tools that cut mainly on one side. This enables the physician to focus the direction of the cut and at the same time, maintain alignment along the longitudinal axis of the implant. Such feature might be particularly useful for careful, “micro-forceps″-assisted removal of scar and calcified tissue through narrow clearances and sensitive organs.

As noted above, it is desirable to combine the insertion and extraction of implants and tissues with a suitable real-time imaging that is easy to understand to facilitate expeditious and inexpensive surgical procedures. Real time imaging provides the ability to accurately locate implants and to insert and manipulate the retrieval unit so that these implants can be accurately inserted and removed with minimal damage to the implant or to surrounding tissue.

Guiding the tips of both the nested catheter needles 12 and 14 to the correct depth necessitates an active imaging technique with great familiarity to clinicians and health care professionals. Stereotactic ultrasound imaging is an affordable and widely used imaging technique for precisely directing the tip of a delicate instrument (such as a needle) in order to reach a specific locus in the body. Typical diagnostic ultrasound for fetal imaging is around 3.5 to 7 MHz, which allows imaging at great depths (tens of centimeters) with limited resolution. High frequency ultrasound (typically in the range of 7 to 50 MHz) is suited for skin imaging with penetration depth of 5 to 15 mm. High frequency ultrasound can easily assess the exact thickness of epidermis and dermis, as well as the size and depth of the subcutaneous tissue, where most miniaturized sensor implants are implanted.

In order to test the efficacy of the nested catheter needles, experiments were conducted on the skin of a commercially available chicken wing. These chicken wings were vacuum packaged into a transparent plastic bag to emulate a tight epidermis. With the use of ultrasound transmission gel applied on top of the transparent plastic membrane a good contact was established between the skin and the probe.

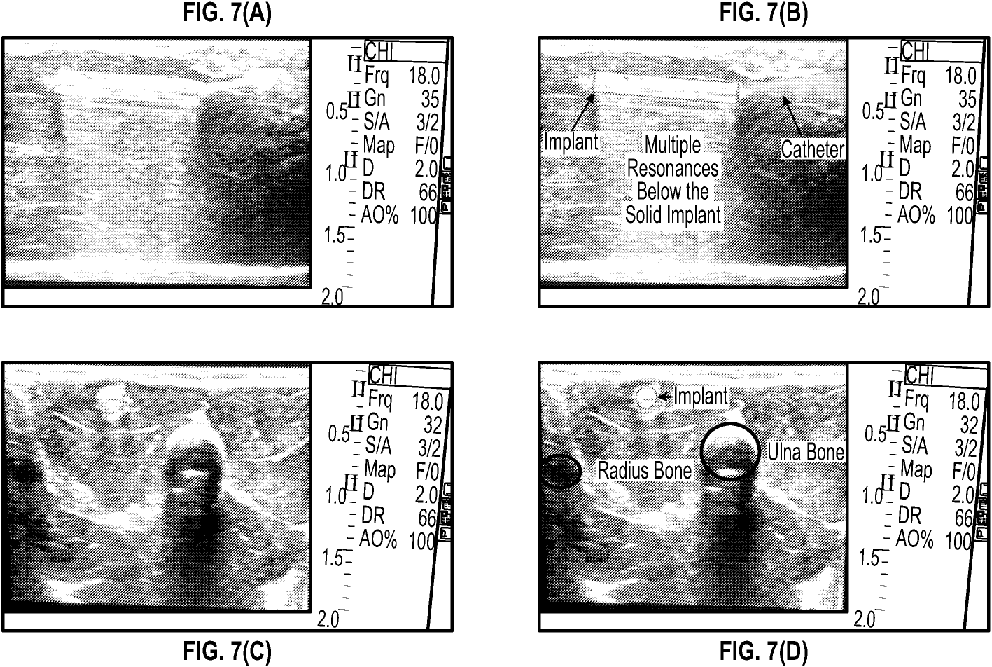

FIGS. 7(A)-7(D) represent a series of photomicrographs that show 12 MHz ultrasound images of an implant and a nearby extraction catheter situated between the ulna and radius bones of the chicken wing. FIGS. 7(A) and 7(C) illustrate two 12 MHz ultrasound images of a 2.1×9 mm implant in the subcutaneous tissue of a chicken wing at about 2.5 mm in depth. This implant was injected parallel to the ulna and radius bones of the chicken wing (FIG. 7(D)) using the insertion tool described in FIG. 3. FIGS. 7(A) and 7(C) depicts the longitudinal (parallel) and transverse (perpendicular) ultrasound images of the implant, respectively. In the longitudinal image, the implant is clearly visible due to the multiple resonances below the solid implant surface (FIG. 7(B). In addition, an approaching catheter is also shown to the right of the implant, which is indicated in FIG. 7(B).

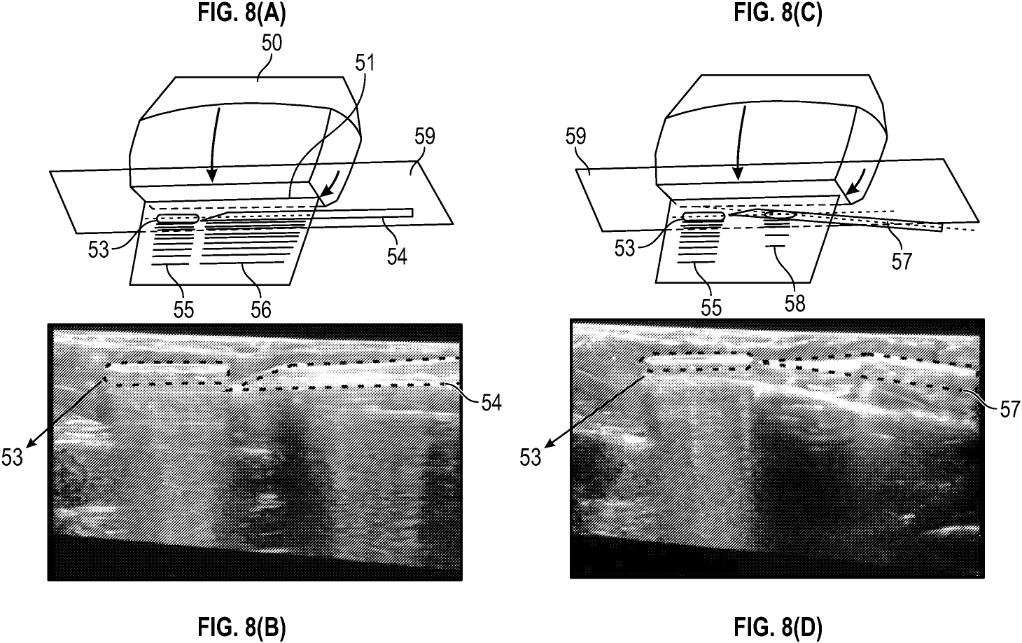

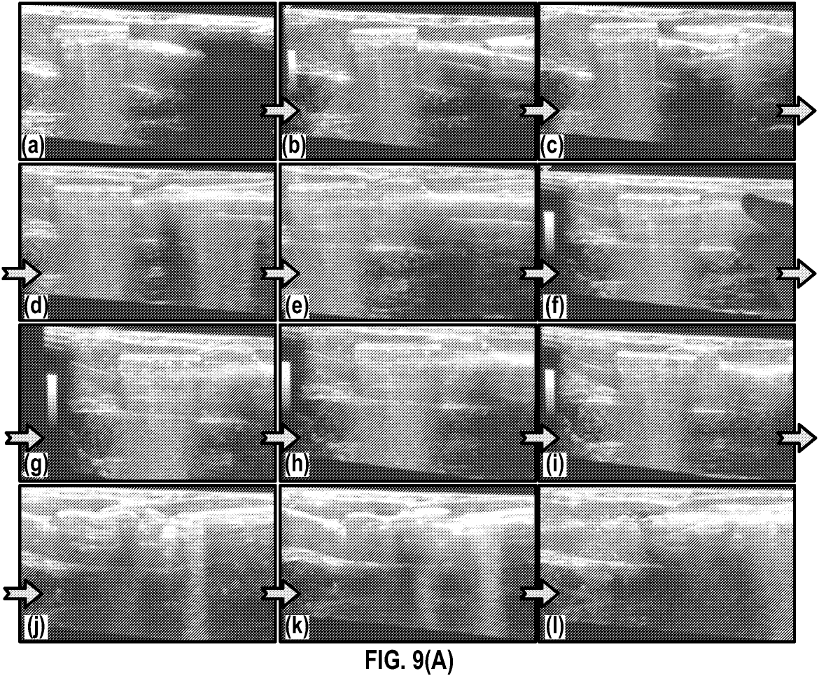

Resonances from ultrasound imaging may be used to facilitate location of the retrieval unit 1002 relative to the implant. As shown in FIGS. 8(A) - 8(D), the multiple ultrasound resonances 55 and 56 below a planar interface of a rigid object (i.e. implant 53 and catheter 54) provide a powerful venue to ensure under skin 59 alignment of these objects within the narrow imaging-plane 51 of an ultrasound probe 50. FIG. 8(A) is a schematic depiction of the multiple resonances 55 and 56 underneath both implant and catheter needles (shown in dotted lines in the FIG. 8(B)) that may be used to indicate alignment of these objects in the ultrasound imaging-plane. FIG. 8(B) is a photomicrograph that corresponds to the schematic depiction of FIG. 8(A) of the location of both implant and catheter that may be used to indicate alignment of these objects with the ultrasound imaging-plane. In FIGS. 8(A) and 8(B), the implant may be seen to be close to the needle(s). The presence of multiple ultrasound resonances for both objects signify that both implant and approaching catheter are well aligned within the ultrasound imaging-plane.

FIG. 8(C) is a schematic depiction of the multiple resonances underneath both implant and catheter that may be used to indicate alignment of these objects in the ultrasound imaging-plane. FIG. 8(D) is a photomicrograph that corresponds to the schematic depiction of FIG. 8(C) of the location of both implant and catheter that may be used to indicate alignment of these objects with the ultrasound imaging-plane. In FIGS. 8(C) and 8(D), the implant may be seen to be further from the needle(s) that in the FIGS. 8(A) and 8(B). In FIG. 8(C), the extraction catheter 57 is intentionally tilted and misaligned away from the ultrasound imaging-plane. Such misalignment eliminates the resonance feature 58, and while the shape of the catheter is still visible, it denotes that the operator must perform corrective actions to re-establish alignment.

Combining imaging with control of the retrieval tool is therefore very useful and valuable. While the FIGS. 7(A) - 7(D) and 8(A) - 8(D) have shown how ultrasound imaging may be combined with the claimed device to insert and extract implants, there are other types of imaging that may be used in conjunction with the device (with or without the ultrasound imaging). Suitable examples of these various types of imaging include visible light imaging, infrared imaging, computer aided tomography, positron emission tomography, xray imaging, electron beam imaging (e.g., scanning electron microscopy), or the like, or a combination thereof. Combinations of these methods with ultrasound imaging can also be undertaken.

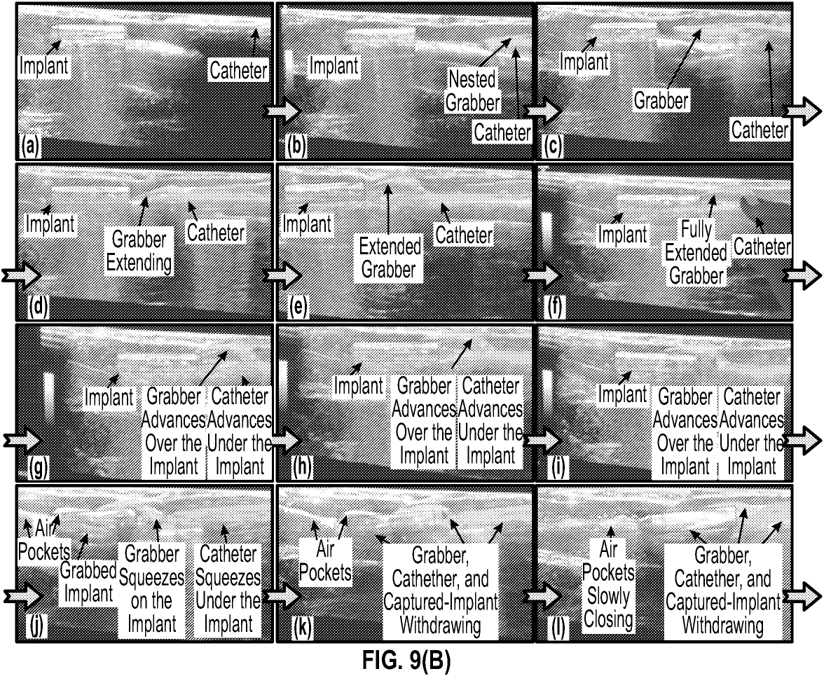

FIG. 9(A) illustrates a sequence of ultrasound captured images (12 MHz) from an extraction video of a 2.1×9 mm implant implanted in the subcutaneous tissue of a chicken wing. FIG. 9(B) provides the corresponding shape-identified and text-annotated items, imaged in FIG. 9(A). The first three images FIG. 9(A)(a-C) and FIG. 9(B)(a-C) illustrate how the fully-nested extractor tip 23 and 24 (needles 12 and 14 from FIGS. 1(A) - 1(D)) (catheter is mainly visible) approaches the implant. The following three images FIG. 9(A)(d-f) and FIG. 9(B)(d-f) show how the grabber extends. In the next three images FIG. 9(A)(g-i) and FIG. 9(B)(g-i) both catheter and grabber (needles 12 and 14 from the FIGS. 1(A) - 1(D)) slowly slide around the implant, prior of squeezing on the implant FIG. 9(A)(j) and FIG. 9(B)(j) and pull the implant backwards and FIG. 9(A)(k, l) and FIG. 9(B)(k, l).

The generation of air pockets behind the squeezed and withdrawn implant are also visible in the images of FIG. 9(A)(j, l) and FIG. 9(B)(j, l). This is because the implant was recently implanted in the subcutaneous tissue of a chicken wing from the opposite side and upon squeezing and pulling the implant to the right, cavities to the left are now made visible. This data indicate that high-frequency ultrasound imaging provides a powerful venue to not only find the location and depth of the implant, but also follow it real time during both the insertion and extraction sequence.

Ultrasound imaging is also important in identifying the skin position, where probe insertion will lead a proper alignment to the longitudinal axis of the miniaturized implant. Ultrasound imaging tools are steadily advancing in terms of cost reduction and image enhancement. Moreover, ultrasound imaging can provide elastography results which can further assist in differentiating between soft and hard tissues particularly around an implant. When elastography and modulus assessment is combined with real-time 3D imaging, it becomes apparent that ultrasound stereotactic guidance can play a critical role in low-cost, minimal-invasive extraction of miniaturized implants.

With this in mind, ultrasound imaging is actively engaged in this invention as a low-cost imaging method among other more expensive and higher resolution stereotactic guiding and imaging methods (i.e., X-ray computed tomography, magnetic resonance imaging, stereotactic radiosurgery, and the like.).

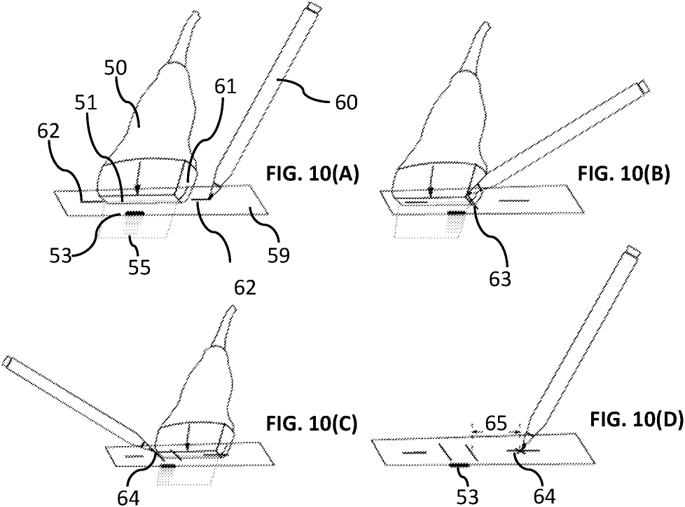

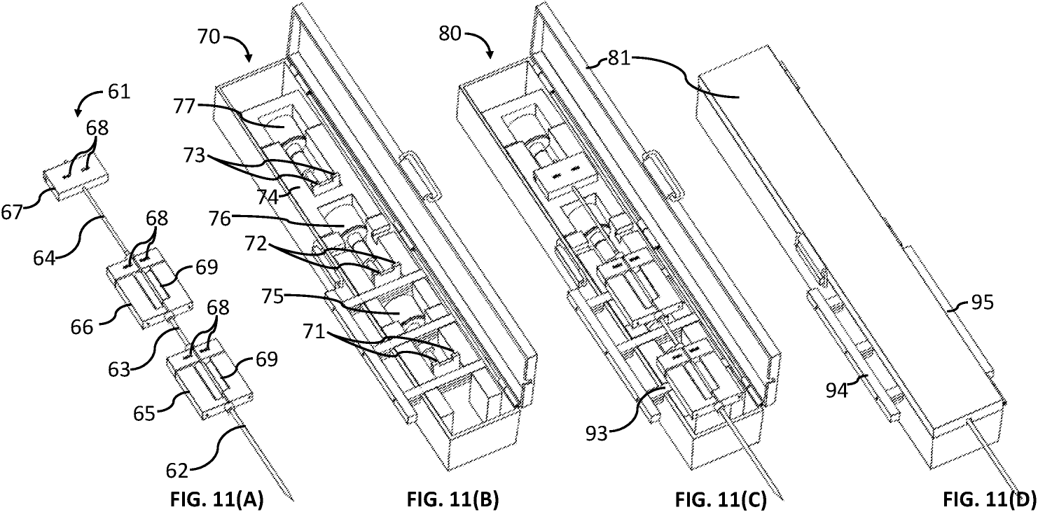

The process of ensuring that is illustrated in FIG. 10. First, the general area of the implant must be identified on the skin 59. Then, as shown in FIG. 10(A) by aligning the ultrasound imaging-plane 51 to that of the implant 53, the generation of multiple ultrasound resonances 55 are witnessed. This signifies that the ultrasound imaging-plane 51, shown at both sides of the probe 50 with either a thin line or a thin arrow 61, guides the operator to mark two small longitudinal lines 62 on the skin 59 with a marker 60. Next the operator moves the ultrasound probe left (FIGS. 10 (B)) and right (FIGS. 10 (C)) while retaining the multiple ultrasound resonances 55 to mark both tips of the implant. Subsequently, the insertion point 64 is identified on one of the two longitudinal lines 62 at a predefined distance 65 from the tip of the implant 53 as shown in FIG. 10(D).

With reference now once again to the FIGS. 1 and 2(A) - 2(D), in order to controllably advance and withdraw the co-axially aligned needles 12 and 14 and plunger 16 of the retriever unit 1002, a battery powered controller unit 1006 has been developed. This battery powered controller unit 1006 is depicted in the FIG. 11 and comprises:
(a) Nested catheter set for extraction (or insertion) (also referred to as the retrieval unit 1002);
(b) A motorized tool that drives the retrieval unit 1002;
(C) Foot-control-unit for hands-free, manual operation; and
(d) Controller unit that houses the:
   (i) Arduino-based logic that controls all fully-automated, semi-automated and manual functions;
   (ii) Stepping-motor microcontrollers;
   (iii) Powering batteries and power recharge circuit to deactivate the Arduino logic while the unit is charging;
   (iv) Build-in display with side-activated buttons to guide the selection of: 1) system initialization (ON/OFF button); 2) system check; 3) battery charge check; 4) motion calibration; 5) selection of insertion or extraction sequence; and 6) display of the relative displacement of the three components of the nested catheter set, which controls their 3D shape and grabbing/releasing ability. These embodiments are depicted in the FIGS. 11(A) -11(D) and each of these figures are detailed below.

FIG. 11(A) illustrates an exemplary embodiment of the nested catheter set (needles 12, 14 and plunger 16 from the FIGS. 1(A) - 1(D)) for insertion or extraction 61. This is made of the co-axially arranged retrieval unit 1002. tool 63 and plunge 64 subcomponents. Each respective subcomponent is equipped with an end-holder, 65, 66, and 67. Each holder is equipped with two slots, 68. These slots fit on the prongs of the corresponding respective handlers 71, 72, and 73, in the insertion/extraction tool box 70 shown in FIG. 11(B). Unlike the plunger, the holders for the retrieval unit 1002 and tool 63 are equipped with a 90° rotation feature 69 described below.

FIG. 11(B) illustrates an exemplary embodiment, of the insertion/extraction tool box 70. This comprises a track 74 housing the three linear stepping motors 75, 76, and 77, to actuate the needles (the jaws) in the retrieval unit 1002. tool, and plunger, respectively. The moving shaft of each motor is attached to the corresponding handler 71, 72, and 73, equipped with two prongs. These prongs fit to the slots of the corresponding holder 68 and afford the controlled linear translation of the needles, tool and plunger, each independently from the other two. FIGS. 11(C) and 11(D) illustrate the assembled extraction tool 80 with open and closed lid 81. The lid and prongs confine the nested catheter set within the z- and y-axis, and upon activation of the corresponding motor, linear movement of the needles of the retrieval unit, tool and plunger is realized along the x-axis.

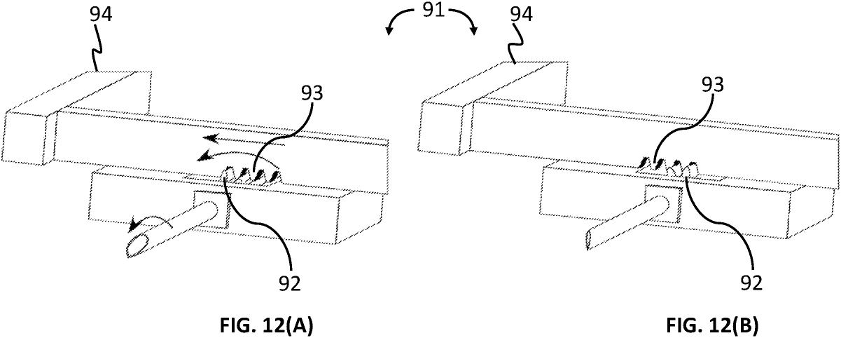

FIGS. 12(A) and 12(B) illustrates an exemplary embodiment of how a 90° rotation is realized for the needles of the retrieval unit and tool 91. For this, both the needles 12 and 14 of the retrieval unit (See FIGS. 1(A) - 1(D)) are equipped with a rotary gear 92. This rotary gear is elongated along its x-axis to ensure engagement for all allowed translations of both needles of the retrieval unit and Tool. The two elongated rotary gears 92 slide on top of two linear gears 93 affixed at the base of linear track 74. On each end, the two linear gears 93 are connected to two handle bars 94 and 95 on opposite sides of the tool. By pressing the left handle bar 94, the linear gear rotates the needles of the retrieval unit needles 90° clockwise, while by pressing the left handle bar 95 a 90° counter clockwise rotation is realized, as shown in FIGS. 12(A) and 12(B). In the FIG. 12(B), it may be seen that the needle 14 is rotated through 90 degrees from its position in the FIG. 12(A) upon moving the left handle bar 94. While the FIGS. 12(A) and 12(B) are hand activated, they may also be activated using miniature stepper motors if desired.

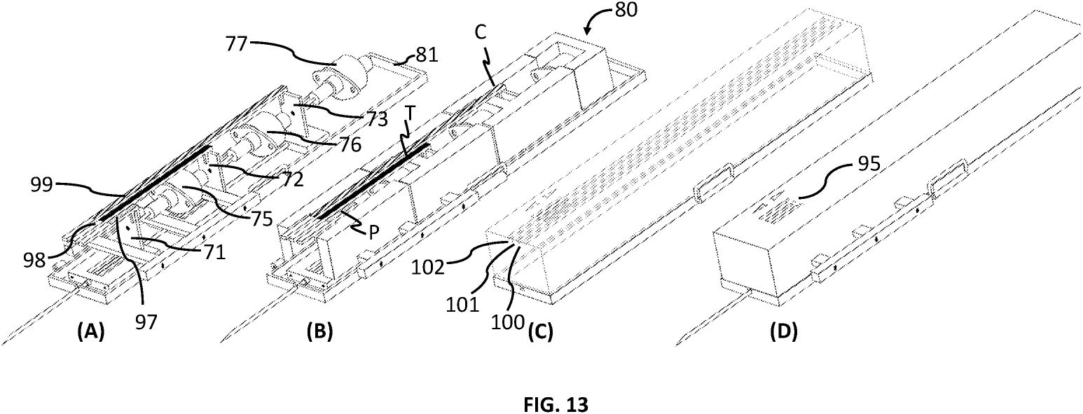

The insertion/extraction tool also provides to the operator a visual gauge 95 in order to assess the x-axis travel of each of three linear motors that translate the three moving elements (i.e. needles 12 and 14 and plunger 16). FIGS. 13(A) - 13(D) illustrates how such visual gauge is implemented in the insertion/extraction tool 80. The catheter (needle 14), tool (needle 12) and plunger 16 are assigned the letter C, T and P on the visual gauge 95, respectively.

FIGS. 13(A) - 13(D) illustrates exemplary embodiments of the opposite view of the insertion/extraction tool 80, with the lid 81 facing down. In FIG. 13(A) the three linear motors 75, 76, and 77 and their corresponding handlers 71, 72 and 73 are readily viewed. FIG. 13(B) depicts an isometric view of the insertion extraction tool with color coded indicator bars marked C (catheter), T (tool) and P (plunger) for the positions of the needles 12 and 14 and the plunger 16. On the topside of these handlers (opposite from the side with the prongs), three notches are situated, where color-coded indicator bars 97, 98, and 99 are affixed with red (shown in hash), gray (shown in black) and blue (shown in sprinkled) patterns for C, T, and P respectively. These color-codded indicator bars are confined between the motor frame and outer case of the insertion/extraction tool and they each reside within individual tracks 100, 101, and 102, shown in FIG. 13(C). The relative translation of C, T, and P can be readily gauged by looking through the three openings on the top of the case (visual gauge 95) as shown in FIG. 13(D). This provides a visual verification to the insertion/extractor operators of the relative C, T, and P travel with respect to each other, which controls the shape and function of the nested-catheter set at any given step.

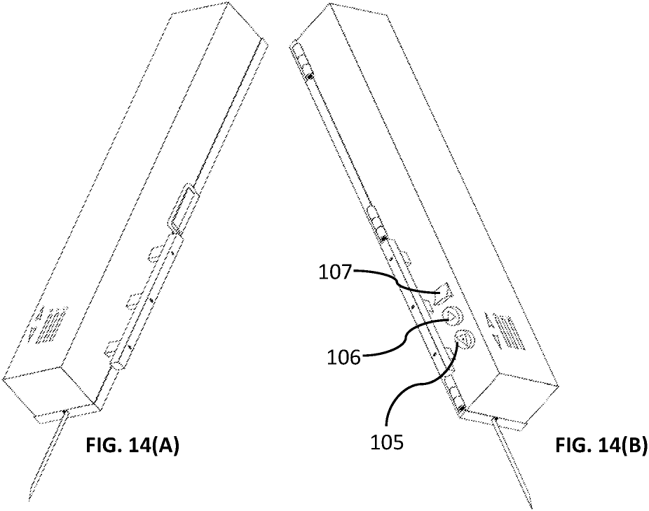

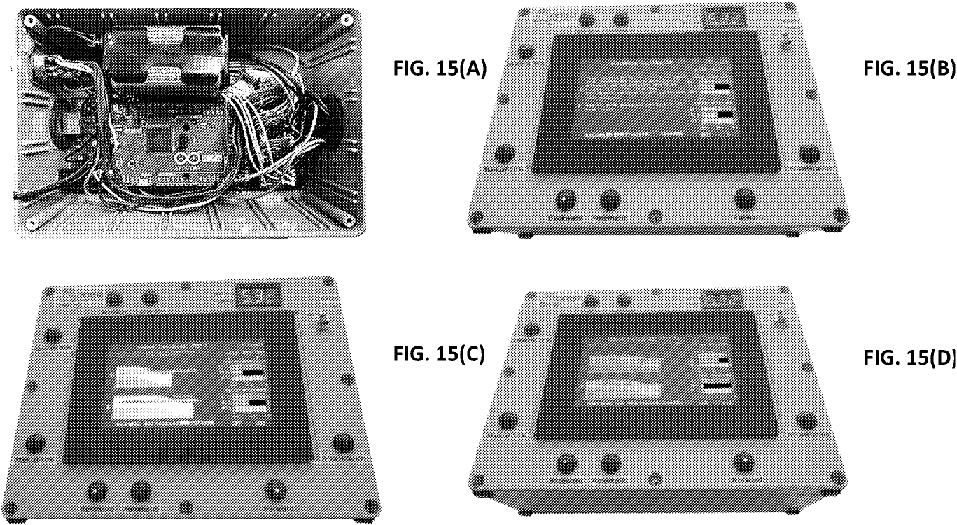

The insertion/extraction tool is also equipped with two advanced buttons (FIG. 14). These buttons interface directly with the Arduino-based logic shown in FIG. 15(A), which controls the automated insertion/extraction program (FIG. 15) that controls the needles 12 and 14 and plunger 16 (See FIGS. 1(A) - 1(D)). FIGS. 14(A) and 14(B) depicts opposite sides of the insertion/extraction tool box. In the FIG. 14(B), the front button with the forward pointing arrow 105 has a center bead for haptic recognition. This button moves the insertion or extraction program forward to the next step. The rear button 106 with the backward pointing arrow and smooth surface moves the insertion or extraction program backwards to the previous step. As described next both automated insertion or extraction program have discrete steps with preset relative positions for C, T, and P. These preset relative C, T, and P positions are displayed on the build-in display of the insertion extraction tool shown in Figure and can be verified by the C, T, and P visual gauge on top of the insertion/extraction tool. The automated forward and backwards button are situated in front of a raised index finger stop 107, where the index finger of the operator would rest. For left-handed operators, the haptic index finger stop 107, as well as the forward 105 and the backwards 106 buttons are located on the on the left side of the insertion/extraction tool.

FIGS. 15(A)-15(D) illustrates the flat panel display of the controller unit for the automated insertion, boring and extraction tool. An Arduino Processor Platform drives all functions of the controller unit as shown in FIG. 15(A). Using custom software the Arduino processor interfaces with the three stepping-motor microcontrollers to generate controlled displacement for the C, T, and P according to the distinct steps that the operator is at, during the insertion or extraction sequences described next. FIG. 15(A) illustrates the battery powered control unit for motorized insertion and extraction hand-tool. FIG. 15(B) illustrates representative captured images of the cover flat panel display describing calibration. FIG. 15(C) illustrates representative captured images of the cover flat panel display detailing implant insertion. FIG. 15(D) illustrates representative captured images of the cover flat panel display detailing boring/extraction.

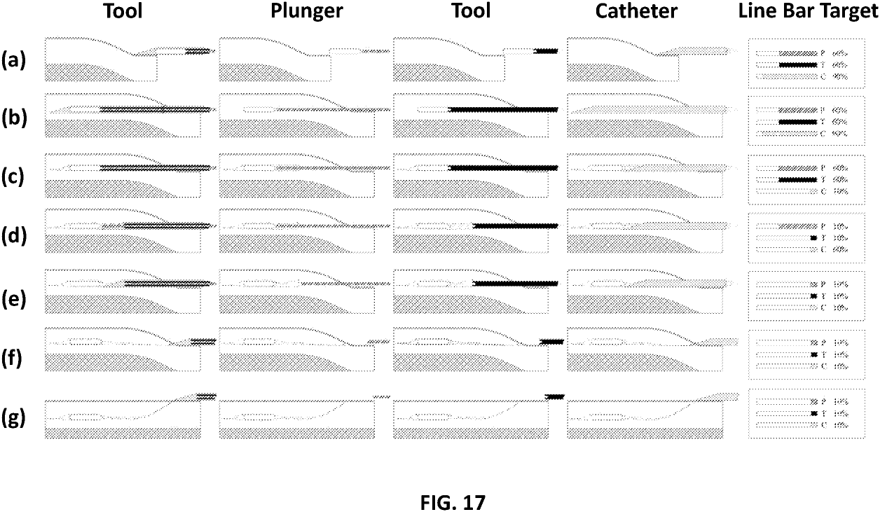

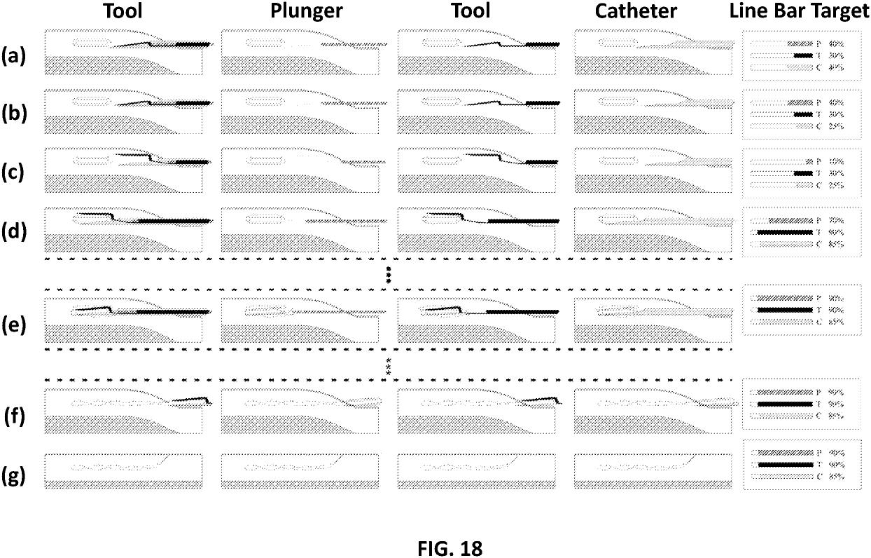

FIGS. 15(B) - 15(D) illustrate the front interface unit of the insertion/extraction control unit together and its flat panel display. The right part of the display, two sets of three line-bars reside that indicate the relative displacement for the C, T, and P linear actuators in three modes: (a) micrometer units displacement (top); (b) percentage mode left, and (C) graphical percentage mode (as color line bars) with one-to-one correspondence to the visual gauge 95 on top of the insertion/extraction tool at FIG. 13(D). The top set of three line bars is interactive (i.e., as the relative displacement for the C, T, and P linear actuators change the displacement, % and line-bar filling is changing). The bottom set of three line-bars indicate the target positions of linear actuators in order to perform the given configuration to the insertion or extraction tool. These target bar positions for the C, T, and P linear actuators are shown in FIG. 17 and FIG. 18, for all steps needs for implant Insertion and Extraction, respectively, and are discussed in greater detail below.

As shown in FIG. 15(A), the controller unit box houses four Ni-MH AA rechargeable batteries, which provide a total of 6 Volts, when fully charged. These four batteries are capable to operate the Arduino-based logic, stepping-motor microcontrollers, build-in display, insertion/extraction tool and the foot-control-unit. When the unit it turned on and the charge is equal or below 40%, the logic prompts the operator to connect the controller unit with the recharger, while at the same time prevents operation. Operation is permitted only if the charge equals or above 70%.

Following controller initialization, the operator is prompted to ensure that the displacement of the C, T, and P linear actuators are properly calibrated. FIG. 15(B) shows the control screen for an automatic calibration step. This step is performed prior to inserting the nested catheter set for insertion or extraction 61 to the hand tool 70, shown in FIG. 11. After the automatic linear actuation calibration step, the operator can also perform a manual calibration step is he/she deems this necessary. Following this, the operator is prompted to select whether to perform an insertion or extraction sequence.

FIG. 15(C) and FIG. 15(D) illustrate one of the many screen shots of the insertion and extraction sequence, respectively. To the left side of the display one can see two images. These images stem directly from insertion/extraction sequences of FIG. 17 and FIG. 18, respectively. The upper image is the tool shape (left) and C, T, and P bar-graph (right) that the operator is starting the given step. The bottom left image is the expected tool shape with respect to the implant after the given step is performed, together with the line target displacement for C, T, and P. In such a manner the operator is actively guided on what each step entails together with the purpose of each step.

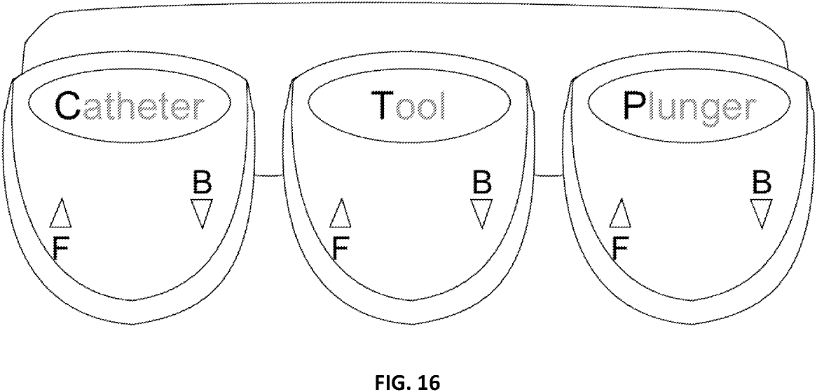

FIG. 16 illustrates the foot control unit. This is intended for hands-free manual operation (forward or backwards) of the catheter “C” (needle 14), tool “T” (needle 12) and plunger “P” 16. The three indicated “F” and “B” allow for independent forward and backward movement for the catheter, tool and plunger, respectively. As described next the manual operation provides also fine tuning, if needed, to the discrete steps of the automated insertion and extraction program that have preset relative positions for C, T, and P.

FIG. 17 illustrates the distinct steps together with the relative plunger 16, tool (needle 12) and catheter (needle 14) displacement (shown in the Line Bar Target) needed to impart the desired shape changes to the overall needle-based micro-forceps (Tool) for the controlled implant insertion according to FIGS. 3(A) - 3(D). FIG. 17 illustrates the distinct steps used to insert a miniaturized implant. First the skin is lifted (by a gentle pinch) and the needle-based inserter, with the implant safely nested within the catheter (needle 14), is implanted at the subcutaneous space (shown in white), situated between the dermis (top) and muscle (bottom shaded region). After the needle-based inserter has reached the predetermined subcutaneous site (Step (b) in FIG. 17), the catheter (needle 14) pulls back to expose the implant (Step (C) in FIG. 17). In Step (d) of FIG. 17, the tool (needle 12) slides back while the plunger 16 remains put. This releases the implant. Subsequently, the plunger 16 is retracted back (Step (e) in FIG. 17) and then the fully nested needle-based inserter, without the implant, pulls back (Step (f) of FIG. 17), before relaxing the gentle pinch and allowing the skin to go back to its original shape (Step (g) of FIG. 17).

FIG. 18 illustrates the distinct steps together with the relative plunger 16, tool (needle 12) and catheter (needle 14) displacement (shown in the Line Bar Target) needed to impart the desired shape changes to the overall needle-based micro-forceps (Tool) for the controlled implant extraction according to FIGS. 1(B) - 1(D) and FIGS. 2(A) - 2(B). FIG. 18 illustrates the distinct steps used to bore the tissue around the miniaturized implantable biosensor and safely extract it. First the implant site, orientation and place of insertion is carefully determined, as explained in FIG. 10. Then the skin is gently lifted and the fully retracted needle-based “micro-forceps” nested extractor is inserted until the tip reaches the bottom tip of the implant (Step (a), FIG. 18). Then the catheter (needle 14 - see FIGS. 1(B) -1(C)) pulls back to expose the grabber 15 (Step (b), FIG. 18). This provides to the grabber 15 the clearance to safely extend upwards by withdrawing the plunger 16 in Step (C), FIG. 18. In Step (d), all three components of the nested-catheter-set move forward around the tissue surrounding the implant (FIG. 18). This allows the first two 90-degree cuts to be performed around the tissue that surrounds the implant, as shown in FIG. 2(A). Subsequently, Step (d) is reversed to Step (C) and the operator is instructed to manual push the percutaneous boring side-bar 94 shown in FIG. 11 and rotate the Catheter and Grabber by 90° and re-perform Step (d) to complete the boring process. In Step (e), the Plunger extends forward and instructs the operator to slightly move the tool and test whether the implant has been arrested (FIG. 18). Once a firm grasp has been accomplished with the implant, the operator should carefully slide out the nested extraction tool and implant and allow the wound to heal.

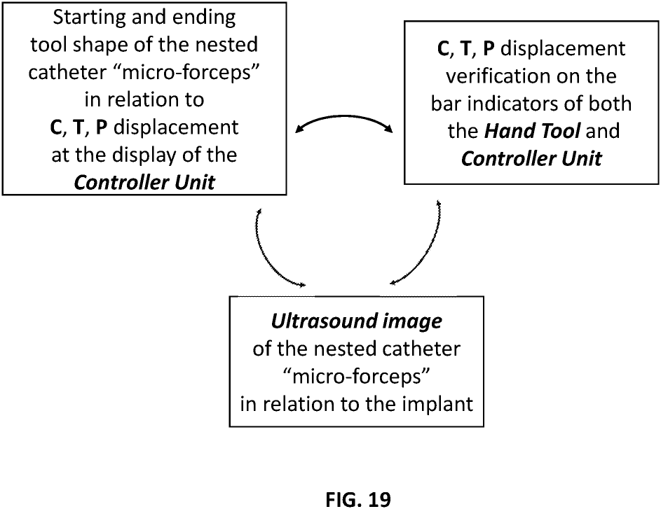

FIG. 19 provides a graphical representation of the visual verification provided to the operator that the insertion/extractor tool functions properly and he/she is actively guided to process and interpret the ultrasound images obtained. This is based on the fact that the C, T, and P travel gauge is intimately linked to the shape of the needle-based “micro-forceps” (at any given step). Moreover, as shown in FIG. 9 the shape of the needle-based “micro-forceps” can be readily deciphered and correlated to the expected tool shape displayed in the Controller unit screen. Correspondingly, the combination of travel gauge verification (on the Controller display and the Hand tool) with nested-catheter set shape-verification (on the Controller display and the Ultrasound images) instills a high degree of confidence on the safety and reproducibility of the insertion/extraction methodology.

If at the end of Step (e), the implant arrest is not secure, the operator is prompted to press once more time the automated forward control button (FIG. 16). This extends the plunger 16 (See FIG. 1(B)) a bit farther to apply higher pressure to the implant and perform a better grip. If still a firm grip has not been established, then the operator is instructed to back rotate the catheter (needle 14) and grabber 15 and ensure that the implant is within the catheter and grabber prongs. This can be performed by reversing back to Step (C) and re-perform Steps (d) and (e), before resorting to manual operation, using the foot-control-unit to further extend the plunger and apply more force to the implant.

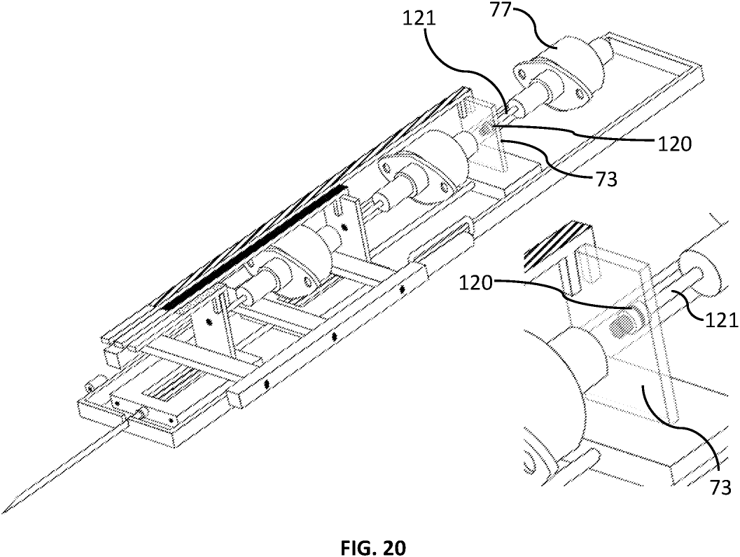

In another embodiment of this invention, the force applied in Step (e) of FIG. 18 can be also assessed and conveyed to the operator. This is intended to prevent applying excessive force to the implant during its arrest, which could possibly break it. FIG. 20 shows that by inserting a force gauge 120 between the handler 73 and the shaft 121 of the linear stepping motor 77 of the Plunger, the force applied to the needle-based “micro-forceps” can be readily assessed.

This necessitates that the controller unit has previous stored the force pattern vs. plunger displacement (along the positions 17, 18, and 19 of FIG. 1) as the plunger slides outwards to close the needle-based “micro-forceps” in the absence of an arrested implant. Subtracting out the stored force pattern from the force pattern with an arrested implant provides the net force applied to the Plunger as part of the needle-based “micro-forceps” squeezing action. Last but not least, in order to assess the actual force applied to the implant, the net force applied to the plunger must be correlated to the net force applied to a calibration force gauge squeezed between the needle-based “micro-forceps”.

This force pattern can be conveyed to the operator via a heat map of the top right plunger displacement line-bar shown in FIGS. 15(B)-15(D). Especially around Step (e) of FIG. 18, the operator can be trained to observe how the color of the Plunger displacement line-bar change with respect to automatic or manual squeeze, indicating that force is applied to the implant.

In another embodiment, an extra dial (indicating force) can be added on top of the already existing three dial line bars of the catheter, tool and plunger shown at the top right of FIGS. 15(B)-15(D).

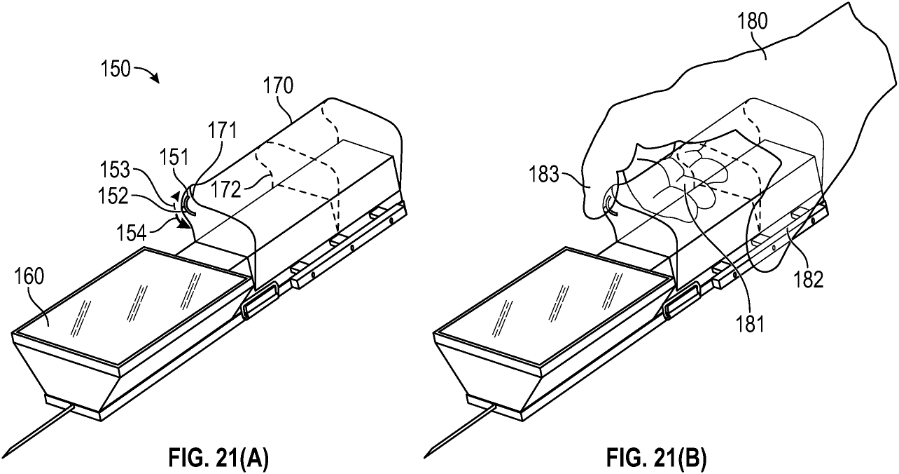

In another embodiment, the forward 105 and backward 106 buttons in FIG. 14 can replaced with a smart rotary dial 151, as shown in FIG. 21. This dial can be equipped in with a spring back feature, where the amplitude of displacement from the rest point is programmed to govern the actuator speed that the given step is completed (i.e. larger downwards 154 or upwards 153 deflection the lesser time it takes to complete a forward or backwards step, respectively). Also on top of the dial there can be a button 152 so that the operator can confirm actuation, as oppose to screen changing that allows him/her to review the steps without actuator movement.

In yet another embodiment, the smart rotary dial 151 could be also outfitted with haptic feedback of the force applied needle-based “micro-forceps”. Such haptic feedback interface is particularly important to assess real time, the force applied in Step (e) of FIG. 18.

Another embodiment of this invention pertains to real-time data fusion, where ultrasound images is juxtaposed to the mechanical motion that change the three-dimensional (3D) configuration of the hand tool.

In typical ultrasound imaging, the medical professional spends considerable amount of his/her time looking at the screen where the ultra-sound image is projected. In doing so, the medical professional is trying to decipher based on the limited resolution and image obstructions/irregularities what he/she is actively seeing. By providing the three-dimensional (3D) local configurations of the tool, the operator can be readily guided in deciphering the ultrasound image and actively coached how to prevent accidental cracking of implant during its arrest and removal.

The first level of data fusion embodiment is to combine the ultrasound monitor with that from the controller unit. This, however, it still requires trained radiologists to operate the tool, who have developed the special skills to watch a monitors, while moving the biopsy tool without visual contact.

The second level of data fusion embodiment involves projecting the as obtained ultrasound images together with displacement-predicted 3D configuration of the needle-based “micro-forceps” right on the actual insertion/extraction hand tool. For this, as shown in FIG. 21, a high-resolution flat panel display 160 can be affixed on the back cover of the insertion/extraction hand tool, where the C, T and P on the visual gauge 95 was originally situated in FIG. 13. This will remove the need for trained radiologists, since all medical professionals possess great eye-to-hand coordination. Combining ultrasound imaging, tool shape, and haptic force feedback at the site of implantation can enable greater adoption to health practitioners and broaden the pool of suitable candidates to be trained on how to use the tool described in this invention.

The added weight that this tool might get by incorporating the flat panel display can be offset by a specially designed hand grip 170. This hand grip 170 is equipped with a special finger recess 172 to enable firm grip. This recess 172 allows the tool to be held via squeezing the palm together with middle and ring fingers 181, while the index finger 183 and thumb 182 are free to perform a variety of tasks. One such task is to operate the aforementioned smart rotary dial 151 situated on an ergonomically curved edge 171.

From the foregoing, it is understood that the invention provides a highly versatile biopsy tool for the controlled percutaneous insertion and extraction of hard implants. This biopsy tool allows the operator to carefully bore around the hard implant that might have developed scar tissue with the surrounding tissue or calcified deposits and extract both implant and surrounding tissue without damaging their local configuration, nor the tool. In addition, this invention provides venues for advance data fusion in order to provide greater situational awareness to the operator. Moreover, by combining ultrasound imaging, tool shape, and haptic force feedback at the site of implantation this invention is geared to enable greater adoption to a larger pool of health practitioners that have good eye-to-hand coordination to use such tool.

In summary, the device 1000 is directed to a percutaneous insertion and extraction catheter tool that places an implant at a predetermined location and extracts it out after some period, comprising two nested catheters and an inner plunger 16; a handheld tool that holds the two nested catheters and the inner plunger; a battery powered controller unit with a flat panel display; and an ultrasound imaging system that guides the catheter tool to the desired location. The two nested catheters comprise an outer catheter (needle 14) and inner catheter (needle 12); wherein the tip of the outer catheter comprises of a sharp-point needle 24 with an elongated step at half height; and wherein the tip of the inner catheter comprises of a sharp-point needle 23 with a triangular serrated cut below, followed by a tubular segment and then a thin bottom segment that is bent upwards to define a flex-operated hinge.

The plunger resides within the inner catheter. The flex-operated hinge of the inner catheter is operated by the sliding in and out of the plunger. The outer and inner catheter create a sharp-tip needle-forceps. The sharp-tip needle-forceps are open when the inner plunger is withdrawn past the flex hinge. The sharp-tip needle-forceps are partially closed when the inner plunger is halfway withdrawn over the flex hinge. The sharp-tip needle-forceps are fully closed and nested within the outer catheter when the inner plunger is directly over the flex hinge.

The outer catheter, inner catheter, and inner plunger are independently actuated with three stepping motors (75, 76, 77), housed within the handheld tool 80, said inner plunger is attached first to a force gauge and then connected with its said stepping motor to obtain force measurement. The outer and inner catheter can rotate 90 degrees with sliding a bar attached to the handheld tool, and said handheld tool has linear bar markers that indicate the relative travel of all said outer catheter, inner catheter, and inner plunger.

The controller unit displays on its screen the relative travel of all said outer catheter, inner catheter, and inner plunger in absolute travel, percentage and linear bar format, and said controller unit has predetermined translation steps for the said outer catheter, inner catheter, and inner plunger for functions including, deploy the said sharp-tip needle-forceps, perform a concentric double-90-degree cut to bore the tissue around the implant, perform a controlled squeeze around the bored tissue to arrest the implant, deploy a nested implant at the desired place with the desired orientation, operate a foot control unit that manually controls and alters all said predetermined translation steps, where the controller unit displays in real-time the shape and configuration of the sharp-tip needle-forceps.

The real-time shape and configuration of the sharp-tip needle-forceps are juxtaposed with the ultrasound images to facilitate the operator. These ultrasound images display multiple resonances underneath both said implant and catheter indicate alignment of these objects in the ultrasound imaging-plane. The ultrasound multiple resonances underneath said implant aid the operator to accurately determine the site of percutaneous catheter insertion, and said controller unit permits operation selected from manual, semi-automatic, and automatic mode chosen by the operator, and said operation is controlled by two forward and backward buttons located at the sides of the handheld unit that actuate the said various steps of the said manual, semi-automatic, and automatic mode chosen by the operator.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

1. A percutaneous catheter apparatus, comprising:
two nested needles; and
an inner plunger; which is guided as a catheter to the tissue surrounding a hard implant to actuate and deploy a pair of sharp-tip needle-forceps that perform two concentric cuts, circularly spaced 90-degree apart from each other, to complete a 360 degree bore around the implant before squeezing to arrest and extract the implant, together with its surrounding tissue.

2. The apparatus of claim 1, where the said guidance is performed by ultrasound imaging.

3. The apparatus of claims 1, where multiple resonances underneath both said implant and said catheter indicate alignment of these objects with the said ultrasound imaging-plane.

4. The apparatus of claims 1, where the said implant and catheter alignment guide the location of the insertion point of the said percutaneous catheter.

5. The apparatus of claim 1, where the said guidance is performed by one selected from infrared imaging, soft X-ray imaging, Magnetic Resonance Imaging, light & ultrasound tomography, stereotactically tomography, computerized axial tomography (CAT).

6. The apparatus of claim 1, where the said implant is a biosensor.

7. The apparatus of claim 1, where the said implant is an RF tag.

8. The apparatus of claim 1, where the said implant is selected one from foreign object, scar tissue, calcinated tissue, hard tumor, hard cyst, ingrown hair/follicle, abnormal bone growth, implanted electrode, implanted catheter fragment.

9. The apparatus of claim 1, where the said two nested needles are able to rotate 90 degrees in order to complete a said 360 degree bore around the said implant.

10. The apparatus of claim 1, where the said 360-degree bored tissue around the implant can be used for biopsy.

11. The apparatus of claim 1, where the said inner rod is equipped with a pressure sensor to monitor the force exerted to the said bored implant during the said squeezing for arresting it together with its surrounding tissue.

12. The apparatus of claim 1, where the said pair of sharp-tip needle-forceps is modified to firmly hold an implant within the said catheter to place it at the desired tissue location and then actuate to position it with the desired orientation.

13. The apparatus of claim 1, where the said two nested needles and the said inner plunger actuate independently from each other in order to deploy the said sharp-tip needle-forceps, then implement the said two concentric 90-degree cuts, and then perform the said squeezing to arrest and extract the implant together with its surrounding tissue.

14. The apparatus of claim 13, where the said independent actuation of the said two nested needles and inner plunger is performed by a handheld tool and its controller unit equipped with a flat panel display.

15. The apparatus of claim 14, where the said handheld tool displays to the operator the relative travel of said actuated two nested needles and inner plunger.

16. The apparatus of claim 14 where the said display shows an image of the said sharp-tip needle-forceps that corresponds to the said relative travel of the two nested needles and inner plunger.

17. The apparatus of claim 14, where the said operation is selected from manual, semi-automatic, and automatic mode chosen by the operator.

18. The apparatus of claim 17, where the said manual mode is enabled by a foot pedal system that operate independently the said actuation of two nested needles and inner plunger.

19. The apparatus of claim 17, where said operation is performed by a sequence of said actuation steps that alter the shape of the said sharp-tip needle-forceps.

20. The apparatus of claim 17, where said operation is performed by a sequence of said actuation steps that alter the shape of the said modified sharp-tip needle-forceps to controllably place an implant at the said desired position and orientation.

21. The apparatus of claim 17, where said operation is aided by a smart rotary dial with press-activation, where speed of completion of the said actuation steps is controlled by the magnitude of dial deflection.

22. The apparatus of claim 21, where and the said sensed pressure opposes said dial deflection.

23. The apparatus of claim 1, where the said handheld tool and its Controller Unit is repeatedly used and after each procedure, the said catheter, comprised of the said two nested needles and an inner plunger, is replaced.

24. A methodology to aid the operator of a percutaneous catheter tool to superimpose the real time shape and position of its sharp-tip needle-forceps to tomographic obtained images, in order to facilitate the safe boring of the tissue surrounding a hard implant prior of extract it together with the arrested implant.

25. A methodology of claim 24, where the said tomographic obtained image is an ultrasound image.

26. The methodology of claim 24 where multiple resonances underneath both said implant and catheter indicate alignment of these objects with the said ultrasound imaging-plane.

27. The methodology of claim 24, where the said implant and catheter alignment guide the location of the insertion point of the said percutaneous catheter.

28. The methodology of claim 24, where the said tomographic obtained images are obtained by one selected from infrared imaging, soft X-ray imaging, Magnetic Resonance Imaging, light & ultrasound tomography, stereotactically tomography, computerized axial tomography (CAT).

29. The methodology of claim 24, where the said implant is a biosensor.

30. The methodology of claim 24, where the said implant is an RF tag.

31. The methodology of claim 24, where the said implant is selected one from foreign objects, scar tissue, calcinated tissue, hard tumors, hard cysts, ingrown hairs/follicles, abnormal bone growth, implanted electrodes, implanted catheter fragments.

32. The methodology of claim 24, where the said sharp-tip needle-forceps are equipped with a pressure sensor to monitor the force exerted to the arrested implant.

33. The methodology of claim 24, where the said sharp-tip, needle-forceps is modified to firmly hold an implant within the said catheter and place it at the desired tissue location with the desired orientation.

34. The methodology of claim 24, where the said methodology is implemented in one operation selected from manual, semi-automatic, and automatic mode chosen by the operator.

35. The methodology of claim 24, where the said superimposition of real time shape of the sharp-tip needle-forceps and tomographic obtained images are projected on a high-resolution display situated on the said percutaneous catheter tool.

36. A percutaneous insertion and extraction catheter tool that places an implant at a predetermined location and extracts it out after some period, comprises of:
two nested catheters and an inner plunger
a handheld tool that holds the two nested catheters and the inner plunger,
a battery powered Controller unit with a flat panel display, and
an ultrasound imaging system that guides the catheter tool to the desired location,
said two nested catheters comprise an outer catheter and inner catheter, wherein
the tip of the outer catheter comprises of a sharp-point needle with an elongated step at half height, and
the tip of the inner catheter comprises of a sharp-point needle with a triangular serrated cut below, followed by a tubular segment and then a thin bottom segment that is bent upwards to define a flex-operated hinge, and
said plunger resides within the inner catheter, and
said flex-operated hinge of the inner catheter is operated by sliding in and out the said plunger,
said outer and inner catheter create a sharp-tip needle-forceps, and
wherein the said sharp-tip needle-forceps are open when the inner plunger is withdrawn past the flex hinge, and
wherein the said sharp-tip needle-forceps are partially closed when the inner plunger halfway withdrawn over the flex hinge, and
wherein the said sharp-tip needle-forceps are fully closed and nested within the outer catheter, when the inner plunger is directly over the flex hinge, and
said outer catheter, inner catheter, and inner plunger are independently actuated with three stepping motors, housed within the handheld tool,
said inner plunger is attached first to a force gauge and then connected with its said stepping motor to obtain force measurement, and
said outer and inner catheter can rotate 90 degrees with sliding a bar attached to the handheld tool, and
said handheld tool has linear bar markers that indicated the relative travel of all said outer catheter, inner catheter, and inner plunger, and
said controller unit displays on its screen the relative travel of all said outer catheter, inner catheter, and inner plunger in absolute travel, percentage and linear bar format, and
said controller unit has predetermined translation steps for the said outer catheter, inner catheter, and inner plunger for functions including,
deploy the said sharp-tip needle-forceps, and
perform a concentric double-90-degree cut to bore the tissue around the implant, and
perform a controlled squeeze around the bored tissue to arrest the implant, and
deploy a nested implant at the desired place with the desired orientation, and
operate a foot control unit that manually controls and alters all said predetermined translation steps, and
said controller unit displays real-time the shape and configuration of the sharp-tip needle-forceps, and
said real-time the shape and configuration of the sharp-tip needle-forceps are juxtaposed to the ultrasound images to facilitate the operator, and
said ultrasound images when display multiple resonances underneath both said implant and catheter indicate alignment of these objects in the ultrasound imaging-plane, and
said ultrasound multiple resonances underneath said implant aid the operator to accurately determine the site of percutaneous catheter insertion, and
said Controller unit permits operation selected from manual, semi-automatic, and automatic mode chosen by the operator, and
said operation is controlled by two forward and backward buttons located at the sides of the handheld unit that actuate the said various steps of the said manual, semi-automatic, and automatic mode chosen by the operator.

37. The tool of claim 36, where the said implant is a biosensor.

38. The tool of claim 36, where the said implant is an RF tag.

39. The tool of claim 36, where the said implant is selected one from foreign objects, scar tissue, calcinated tissue, hard tumors, hard cysts, ingrown hairs/follicles, abnormal bone growth, implanted electrodes, implanted catheter fragments.

40. The tool of claim 36, where the said 360-degree bored tissue around the implant can be used for biopsy.

41. The tool of claims 36, where said operation is aided by a smart rotary dial with press-activation, where speed of completion of the said actuation steps is controlled by the magnitude of dial deflection.

42. The tool of claims 36, where and the said sensed pressure opposes said dial deflection.

43. The tool of claims 36, where the said handheld tool and its Controller Unit is repeatedly used and after each procedure, the said catheter, comprised of the said two nested needles and an inner plunger, is replaced.

44. The tool of claim 36, where the said ultrasound images and the shape and configuration of the sharp-tip needle-forceps are superimposed on a high-resolution display situated on the handheld tool.

45. The tool of claim 36, where the said handheld tool is equipped with an ergonomic handle that can be firmly held between the palm and the middle and ring finger, while both index and thump are free to select and operate between various buttons and rotary dials.
